(19) United States
(12) Patent Application Publication
(10) Pub. No.: US 2025/0383318 A1
(43) Pub. Date: Dec. 18, 2025

(54) SENSOR ELEMENT AND GAS SENSOR

(71) Applicant: NGK INSULATORS, LTD., Nagoya-shi (JP)

(72) Inventors: Yuki ISHIHARA, Nagoya-shi (JP); Takashi HINO, Kasugai-shi (JP)

(21) Appl. No.: 19/234,334

(22) Filed: Jun. 11, 2025

(30) Foreign Application Priority Data
Jun. 13, 2024 (JP) 2024-095787

(51) Int. Cl. G01N 27/407; G01N 27/41; G01N 33/00

(52) U.S. Cl. CPC G01N 27/4072; G01N 27/41; G01N 33/0027

(57) ABSTRACT

A sensor element for detecting a concentration of a specific gas in a measurement-object gas, the sensor element includes: the element body having a measurement-object gas flow section inside that introduces the measurement-object gas and causes the measurement-object gas to flow in a first direction; a diffusion control section that is located in the measurement-object gas flow section, wherein the diffusion control section includes a porous body and a space portion that are provided in at least part of the diffusion control section in the first direction, the porous body is provided so as to occupy a part of a cross-section of the diffusion control section perpendicular to the first direction and allows the measurement-object gas to pass therethrough, and the space portion includes a part of the cross-section not occupied by the porous body and allows the measurement-object gas to pass therethrough.

### CROSS-REFERENCE TO RELATED APPLICATION

The present application claims priority from Japanese application JP2024-095787, filed on Jun. 13, 2024, the content of which is hereby incorporated by reference into this application.

### BACKGROUND OF THE INVENTION

### 1. Field of the Invention

The present invention relates to a sensor element and a gas sensor.

### 2. Description of the Related Art

Hitherto, a gas sensor that detects the concentration of a specific gas, such as NOx, in a measurement-object gas, such as the exhaust gas of an internal combustion engine, is known. For example, PTL 1 describes a gas sensor comprising a sensor element that comprise an element body, a main pump cell, an auxiliary pump cell, and a measurement pump cell. The element body includes an oxygen-ion-conductive solid electrolyte layer and has a measurement-object gas flow section inside that introduces the measurement-object gas and causes the measurement-object gas to flow. The main pump cell includes an inner main pump electrode provided in a first internal cavity of the measurement-object gas flow section and an outer main pump electrode provided on an outside of the element body that is exposed to the measurement-object gas. The auxiliary pump cell including an inner auxiliary pump electrode provided in a second internal cavity located downstream of the first internal cavity in the measurement-object gas flow section and an outer auxiliary pump electrode provided on an outside of the element body that is exposed to the measurement-object gas. The measurement pump cell includes an inner measurement pump electrode provided in a measurement chamber located downstream of the second internal cavity in the measurement-object gas flow section and an outer measurement pump electrode provided on an outside of the element body that is exposed to the measurement-object gas. When using this sensor element to detect NOx concentrations, the oxygen concentration in the measurement-object gas is adjusted in the first internal cavity and the second internal cavity by the main pump cell and the auxiliary pump cell. Next, NOx in the measurement-object gas with the oxygen concentration adjusted is reduced in the measurement chamber. The NOx concentration in the measurement-object gas is then detected based on the pump current that flows when the measurement pump cell pumps out the oxygen produced by the reduction of NOx.

### CITATION LIST

### Patent Literature

PTL 1: Japanese Unexamined Patent Application Publication No. 2017-181499 A

### SUMMARY OF THE INVENTION

In such a gas sensor, even if a composition of the measurement-object gas is the same, an operation of a pump cell (e.g. a value of a pump current flowing through a pump cell of the sensor element) may differ if a dynamic pressure of the measurement-object gas differs. As a result, the dynamic pressure of the measurement-object gas may affect the accuracy in detection of the specific gas concentration. Therefore, it is desirable to reduce the effect of the dynamic pressure of measurement-object gas on the pump cell.

The present invention has been devised to solve such a problem, and it is a main object to reduce the effect of the dynamic pressure of measurement-object gas on the pump cell.

The present invention employs the following device to achieve the above-described main object.

[1] A sensor element according to the present invention is a sensor element for detecting a concentration of a specific gas in a measurement-object gas, the sensor element comprising: an element body having an oxygen-ion-conductive solid electrolyte layer, and having a measurement-object gas flow section inside that introduces the measurement-object gas from a gas inlet and causes the measurement-object gas to flow in a first direction; A pump cell including an inner electrode provided in an internal cavity of the measurement-object gas flow section and an outer electrode provided on an outer surface of the element body, and a diffusion control section that is located upstream of the internal cavity in the measurement-object gas flow section and that provides the measurement-object gas introduced from the gas inlet with diffusion resistance, wherein the diffusion control section includes a porous body and a space portion that are provided in at least a part of the diffusion control section in the first direction, the porous body is provided so as to occupy a part of a cross-section of the diffusion control section perpendicular to the first direction and allows the measurement-object gas to pass therethrough, and the space portion includes a part of the cross-section not occupied by the porous body and allows the measurement-object gas to pass therethrough.

This sensor element has the diffusion control section, which is a part of the measurement-object gas flow section that causes the measurement-object gas to flow in the first direction. The diffusion control section includes the porous body and the space portion that are provided in at least a part of the diffusion control section in the first direction. The porous body is provided so as to occupy a part of a cross-section of the diffusion control section perpendicular to the first direction. The space portion includes a part of the cross-section not occupied by the porous body. As a result, it is possible to make a flow velocity of the measurement-object gas passing through the porous body different from a flow velocity of the measurement-object gas passing through the space portion in the cross-section of the diffusion control section perpendicular to the first direction. Thereby it is possible to cause turbulence in the measurement-object gas after passage and increase a pressure loss of the measurement-object gas. Therefore, the effect of the dynamic pressure of measurement-object gas on the pump cell including the inner electrode that is located downstream of a diffusion control section can be reduced.

[2] In the above-described sensor element (the sensor element according to [1] above), the porous body may be provided in the diffusion control section so as to occupy 30% to 70% of the cross-section.

[3] In the above-described sensor element (the sensor element according to [1] or [2] above), a porosity of the porous body may be 15% or less. In this way, the flow velocity of the measurement-object gas passing through the porous body can be made more different from the flow velocity of the measurement-object gas passing through the space portion, and the pressure loss of the measurement-object gas after passage can be further increased.

[4] In the above-described sensor element (the sensor element according to any one of [1] to [3] above), the porous body may be provided in a plurality so as to form parallel flow paths in the cross-section.

[5] In the above-described sensor element (the sensor element according to any one of [1] to [4] above), the element body may have a longitudinal direction, and may be a layered body that is composed of multiple layers stacked in a layered direction perpendicular to the longitudinal direction, a shape of the cross-section of the space portion may be a predetermined shape with a longer outer circumference than a reference rectangle, and the reference rectangle may be a rectangle that is the same area as the cross-section of the space portion, and the reference rectangle may be a rectangle that is formed by deforming the circumscribed rectangle of the cross-section, which is composed of two sides parallel to the layered direction and two sides parallel to a second direction that is perpendicular to the layered direction, so that short sides become shorter while maintaining the length of long sides, or a rectangle that is formed by deforming the circumscribed rectangle while maintaining a ratio of each side, or a rectangle that is formed by changing the length of each side of the circumscribed rectangle so as to maximize the overlapping area with the cross-section. In this way, the part of the space portion of the diffusion control section having the shape of the cross-section perpendicular to the first direction that is the predetermined shape has a smaller hydraulic diameter than the reference rectangle. Thus, the pressure loss of the measurement-object gas passing through the space portion becomes higher. Therefore, by this shape also, the effect of the dynamic pressure of measurement-object gas on the pump cell including the inner electrode that is located downstream of a diffusion control section can be reduced.

[6] In the above-described sensor element (the sensor element according to [5] above), the outer circumference of the predetermined shape may include a first portion and a second portion that face each other in the layered direction and extend along the second direction, respectively, and at least a part of the first portion and/or at least a part of the second portion may each have a curved shape.

[7] In the above-described sensor element (the sensor element according to [5] or [6] above), the outer circumference of the predetermined shape may include a first portion and a second portion that face each other in the layered direction and extend along the second direction, respectively, and at least a part of the first portion and/or at least a part of the second portion may each have a polygonal line shape.

[8] In the above-described sensor element (the sensor element according to [6] or [7] above), the first portion and the second portion may each have a convex shape on the same side in the layered direction.

[9] In the above-described sensor element (the sensor element according to [6] or [7] above), the first portion and the second portion may each have a convex shape toward a side where they approach each other or toward a side where they are separated from each other in the layered direction.

[10] In the above-described sensor element (the sensor element according to any one of [5] to [9] above), the diffusion control section may have a plurality of the space portions, and each of the plurality of the space sections may have a shape of the cross-section that is the predetermined shape.

[11] In the above-described sensor element (the sensor element according to any one of [1] to [10] above), the diffusion control section may be formed such that a cross-sectional area of a cross-section perpendicular to the first direction is smaller in at least a part of a downstream side of upstream end than in the upstream end. This increases the pressure loss of the measurement-object gas passing through a part of the space portion having a smaller cross-sectional area than the upstream end. Therefore, by this shape also, the effect of the dynamic pressure of measurement-object gas on the pump cell including the inner electrode that is located downstream of a diffusion control section can be reduced.

[12] In the above-described sensor element (the sensor element according to [11] above), the diffusion control section may have an outline of cross-section parallel to the first direction, and the outline may extend so as to curve with respect to the first direction.

[13] A gas sensor according to the present invention comprises the sensor element according to any one of [1] to [12] above. Thus, the gas sensor produces the same advantageous effects as those produced by the above sensor element. For example, an advantageous effect is the reduction of the effect of the dynamic pressure of measurement-object gas on the pump cell.

### BRIEF DESCRIPTION OF THE DRAWINGS

FIG. 1 is a cross-sectional schematic view schematically showing an example of a configuration of a gas sensor 100.

FIG. 2 is a view along the arrow A in FIG. 1.

FIG. 3 is a cross-sectional view taken along the line B-B in FIG. 1.

FIG. 4 is a block diagram showing an electrical connection relationship between a control apparatus 95 and relevant elements including cells.

FIG. 5 is a cross-sectional view taken along the line C-C in FIG. 3.

FIGS. 6A to 6C are partial cross-sectional views showing a manufacturing process of a diffusion control section 11a.

FIG. 7 is an explanatory diagram showing a diffusion control section 11a according to a modification.

FIG. 8 is an explanatory diagram showing a diffusion control section 11a according to a modification.

FIG. 9 is an explanatory diagram showing a reference rectangle S1.

FIG. 10 is an explanatory diagram showing a reference rectangle S2.

FIGS. 11A to 11C are partial cross-sectional views showing a manufacturing process of the diffusion control section 11a in FIG. 8.

FIG. 12 is an explanatory diagram showing a diffusion control section 11a according to a modification.

FIG. 13 is an explanatory diagram showing a diffusion control section 11a according to a modification.

FIG. 14 is an explanatory diagram showing a diffusion control section 11a according to a modification.

FIG. 15 is an explanatory diagram showing a diffusion control section 11a according to a modification.

FIG. 16 is an explanatory diagram showing a diffusion control section 11a according to a modification.

FIG. 17 is an explanatory diagram showing a diffusion control section 11a according to a modification.

FIG. 18 is an explanatory diagram showing a diffusion control section 11a according to a modification.

FIG. 19 is an explanatory diagram showing a diffusion control section 11a according to a modification.

FIG. 20 is an explanatory diagram showing a diffusion control section 11a according to a modification.

FIG. 21 is an explanatory diagram showing a diffusion control section 11a according to a modification.

FIG. 22 is an explanatory diagram showing a diffusion control section 11a according to a modification.

FIG. 23 is a cross-sectional schematic view showing a first diffusion control section 211 according to a modification.

FIG. 24 is a cross-sectional view taken along the line D-D in FIG. 23.

FIG. 25 is a cross-sectional view taken along the line E-E in FIG. 24.

### DETAILED DESCRIPTION OF THE INVENTION

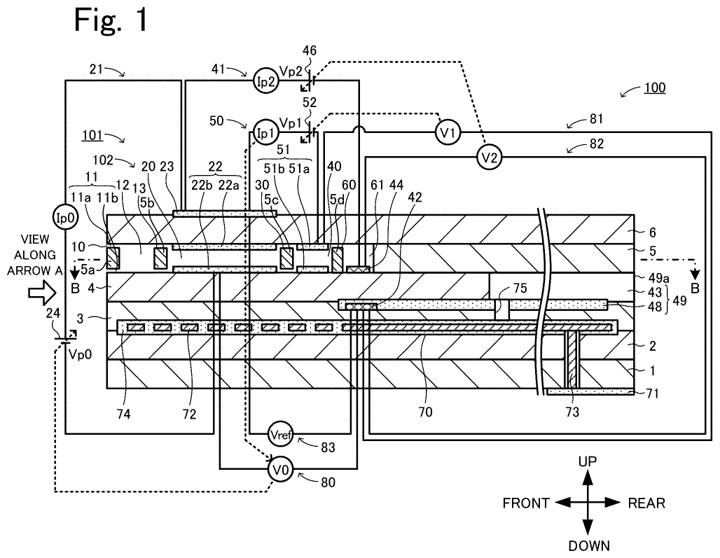

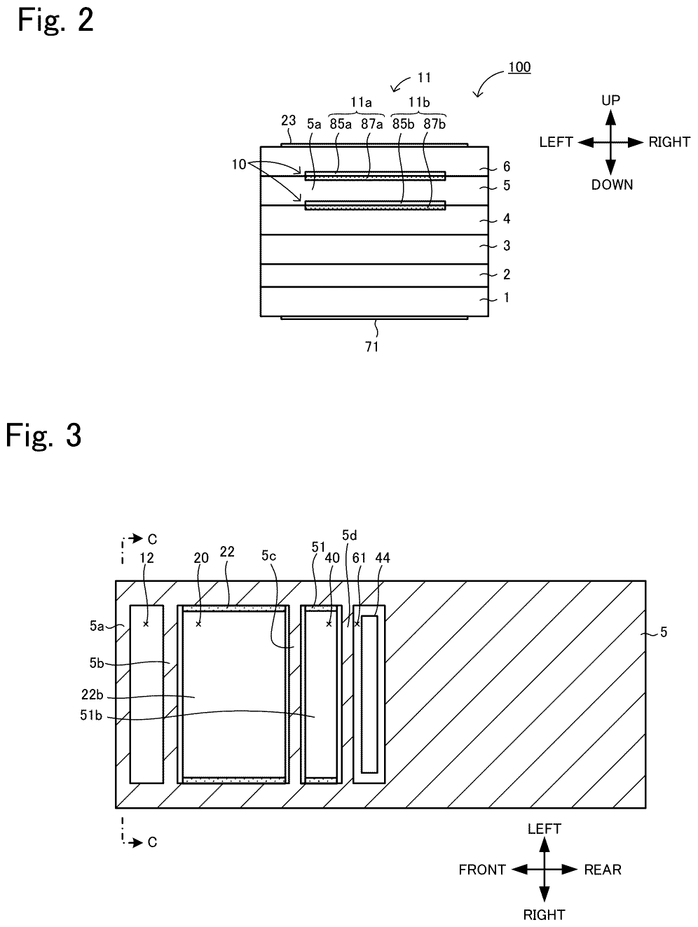

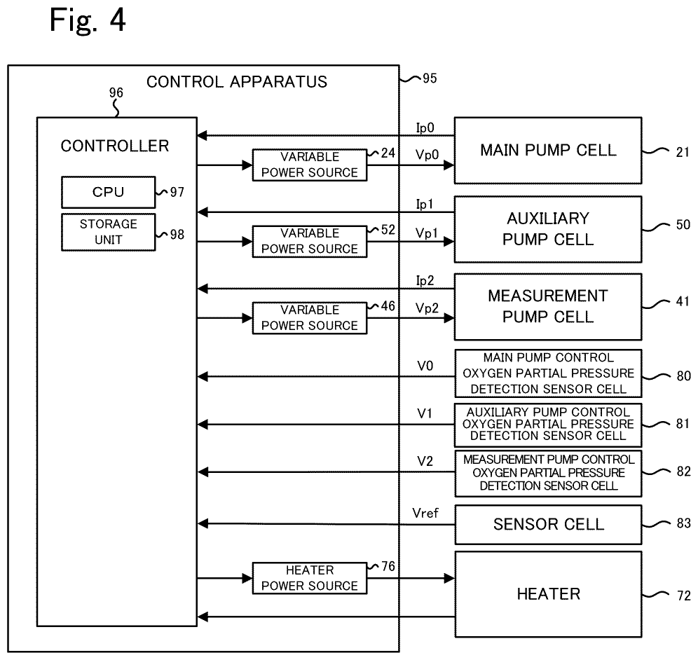

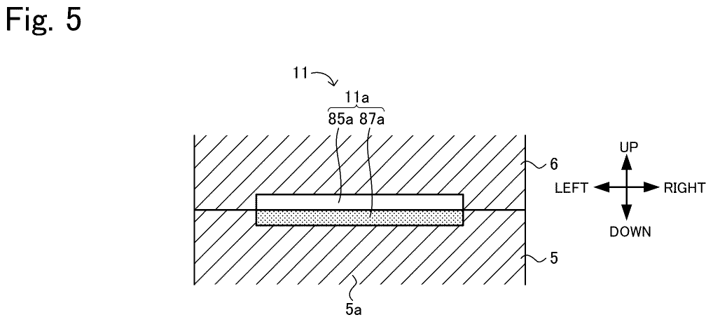

Next, an embodiment of the present invention will be described using the drawings. FIG. 1 is a cross-sectional schematic view schematically showing an example of the configuration of gas sensor 100 which is an embodiment of the present invention. FIG. 2 is a view along the arrow A in FIG. 1. FIG. 3 is a cross-sectional view taken along the line B-B in FIG. 1. FIG. 4 is a block diagram showing an electrical connection relationship between a control apparatus 95, cells and a heater 72. FIG. 5 is a cross-sectional view taken along the line C-C in FIG. 3. FIG. 5 is a cross-sectional schematic view schematically showing an area around a first space portion 85a of a first diffusion control section 11. The gas sensor 100 is installed in a pipe, such as an exhaust gas pipe of an internal combustion engine, for example. The gas sensor 100 uses the exhaust gas from an internal combustion engine as the measurement-object gas, and detects specific gas concentration which is the concentration of a specific gas such as NOx or ammonia in the measurement-object gas. In the present embodiment, the gas sensor 100 measures a NOx concentration as the specific gas concentration. The gas sensor 100 has a sensor element 101 including a long rectangular parallelepiped element body 102, cells 21, 41, 50, 80 to 83 included in the sensor element 101, a heater portion 70 provided inside the sensor element 101, and a control apparatus 95 that includes variable power supplies 24, 46, 52 and a heater power source 76, and controls the entire gas sensor 100. In the present embodiment, as shown in FIGS. 1 to 3, the longitudinal direction of the element body 102 of the sensor element 101 is defined as the front-rear direction (length direction), the layered direction (thickness direction) of each layer 1 to 6 of the element body 102 is defined as the up-down direction, and the direction perpendicular to the front-rear direction and the up-down direction is defined as the left-right direction (width direction). FIG. 5 is illustrated with the up-down length stretched out for the sake of illustration, compared to FIG. 2.

The element body 102 is a layered body in which six layers, that is, a first substrate layer 1, a second substrate layer 2, a third substrate layer 3, a first solid electrolyte layer 4, a spacer layer 5, and a second solid electrolyte layer 6, each made up of an oxygen-ion-conductive solid electrolyte layer made of zirconia (ZrO₂) or the like, are laminated in this order from a lower side in the drawing. The solid electrolyte forming these six layers is a dense, airtight one. The element body 102 is manufactured by, for example, applying predetermined processing, printing of a circuit pattern, and the like on a ceramic green sheet corresponding to each layer, then laminating those sheets, and further firing the sheets to be integrated.

At a tip end portion side of the sensor element 101 (the element body 102) (left end portion side in FIG. 1), a gas inlet port 10, a first diffusion control section 11, a buffer space 12, a second diffusion control section 13, a first internal cavity (an oxygen concentration adjustment chamber) 20, a third diffusion control section 30, a second internal cavity (then oxygen concentration adjustment chamber) 40, a fourth diffusion control section 60, and a third internal cavity (a measurement chamber) 61, are formed adjacent to each other so as to communicate with each other in this order between the lower surface of the second solid electrolyte layer 6 and the upper surface of the first solid electrolyte layer 4.

The gas inlet port 10, the buffer space 12, the first internal cavity 20, the second internal cavity 40, and the third internal cavity 61 are spaces of which top parts, bottom parts, and side parts, provided by hollowing the spacer layer 5, are respectively defined by the lower surface of the second solid electrolyte layer 6, the upper surface of the first solid electrolyte layer 4, and the side surface of the spacer layer 5 inside the sensor element 101.

Each of the first diffusion control section 11, the second diffusion control section 13, and the third diffusion control section 30 is provided as two laterally long slits (openings of which the longitudinal direction is a direction perpendicular to the drawing). FIGS. 2 and 5 illustrate the shapes of the diffusion control section 11a and diffusion control section 11b which constitute the two slits of the first diffusion control section 11. The fourth diffusion control section 60 is provided as a single laterally long slit (an opening of which the longitudinal direction is a direction perpendicular to the drawing) formed as a clearance from the lower surface of the second solid electrolyte layer 6. A part from the gas inlet port 10 to the third internal cavity 61 is also referred to as measurement-object gas flow section.

The sensor element 101 (element body 102) includes a reference-gas introduction portion 49 that allows the reference gas to flow from outside the sensor element 101 to a reference electrode 42 in the measurement of NOx concentration. The reference-gas introduction portion 49 has a reference-gas introduction space 43 and a reference-gas introduction layer 48. The reference gas introduction space 43 is a space that is provided inward from a rear end face of the sensor element 101. The reference-gas introduction space 43 is provided at a position between the upper surface of the third substrate layer 3 and the lower surface of the spacer layer 5 and has lateral sides defined by the side surfaces of the first solid electrolyte layer 4. The reference-gas introduction space 43 has an opening at the rear end face of the sensor element 101. This opening functions as an entrance 49a of the reference-gas introduction portion 49. The reference gas is introduced into the reference-gas introduction space 43 through the entrance 49a. The reference-gas introduction portion 49 introduces the reference gas to the reference electrode 42 while applying a predetermined diffusion resistance to the reference gas received through the entrance 49a. In the present embodiment, the reference gas is ambient air.

The reference-gas introduction layer 48 is disposed between the upper surface of the third substrate layer 3 and the lower surface of the first solid electrolyte layer 4. The reference-gas introduction layer 48 is a porous body composed of a ceramic material such as alumina. A part of the upper surface of the reference-gas introduction layer 48 is exposed in the reference-gas introduction space 43. The reference-gas introduction layer 48 is provided over the reference electrode 42. The reference-gas introduction layer 48 allows the reference gas to flow from the reference-gas introduction space 43 to the reference electrode 42.

The reference electrode 42 is an electrode formed in such a manner in which the reference electrode 42 is sandwiched by the upper surface of the third substrate layer 3 and the first solid electrolyte layer 4. As described above, the reference gas inlet layer 48 that communicates with the reference gas inlet space 43 is provided around the reference electrode 42. As will be described later, it is possible to measure an oxygen concentration (oxygen partial pressure) in the first internal cavity 20, an oxygen concentration (oxygen partial pressure) in the second internal cavity 40, and an oxygen concentration (oxygen partial pressure) in the third internal cavity 61 by using the reference electrode 42. The reference electrode 42 is formed as a porous cermet electrode (for example, a cermet electrode of Pt and ZrO₂).

In the measurement-object gas flow section, the gas inlet port 10 is a portion that is open to an external space, and a measurement-object gas is taken into the sensor element 101 from the external space through the gas inlet port 10. The first diffusion control section 11 is a portion that applies predetermined diffusion resistance to a measurement-object gas taken in through the gas inlet port 10. In the present embodiment, a front end of the first diffusion control section 11 is the gas inlet 10. As illustrated in FIGS. 1 and 2, the diffusion control section 11a of the first diffusion control section 11 is provided between an upper surface of a first bridging portion 5a of the spacer layer 5 and the bottom surface of the second solid electrolyte layer 6. The diffusion control section 11b is provided between a bottom surface of the first bridging portion 5a and the upper surface of the first solid electrolyte layer 4. As illustrated in FIG. 3, the first bridging portion 5a is a portion that bridges to the left and right, and that located in a front side of the buffer space 12, which is a portion of the spacer layer 5 that has been punched out. The buffer space 12 is a space provided to guide the measurement-object gas introduced from the first diffusion control section 11 to the second diffusion control section 13. The second diffusion control section 13 is a portion that applies predetermined diffusion resistance to the measurement-object gas introduced from the buffer space 12 into the first internal cavity 20. As illustrated in FIGS. 1 and 2, the second diffusion control section 13 is provided between an upper surface of a second bridging portion 5b of the spacer layer 5 and the lower surface of the second solid electrolyte layer 6, and between a lower surface of the second bridging portion 5b and the upper surface of the first solid electrolyte layer 4. As illustrated in FIG. 3, the second bridging portion 5b is a portion that bridges to the left and right, and that located between the buffer space 12 and the first internal cavity 20, which are portions of the spacer layer 5 that has been punched out. When the measurement-object gas is introduced from the outside of the sensor element 101 into the first internal cavity 20, the measurement-object gas rapidly taken into the sensor element 101 through the gas inlet port 10 due to pressure fluctuations of the measurement-object gas in the external space (due to pulsation of exhaust pressure when the measurement-object gas is the exhaust gas of an automobile) is not directly introduced into the first internal cavity 20 but, after pressure fluctuations of the measurement-object gas are cancelled out through the first diffusion control section 11, the buffer space 12, and the second diffusion control section 13, the measurement-object gas is introduced into the first internal cavity 20. With this configuration, pressure fluctuations of the measurement-object gas introduced into the first internal cavity 20 are almost ignorable. The first internal cavity 20 is provided as a space used to adjust an oxygen partial pressure in the measurement-object gas introduced through the second diffusion control section 13. The oxygen partial pressure is adjusted by the operation of a main pump cell 21.

The main pump cell 21 is an electrochemical pump cell constituted of an inner pump electrode 22 including a ceiling electrode portion 22a disposed on the lower surface of the second solid electrolyte layer 6 over substantially the entirety of an area that faces the first internal cavity 20; an outer pump electrode 23 disposed on the upper surface of the second solid electrolyte layer 6 over an area that corresponds to the ceiling electrode portion 22a in such a manner as to be exposed to the outside of the sensor element 101; and the second solid electrolyte layer 6, the spacer layer 5, and the first solid electrolyte layer 4 that form a current path between the electrodes 22 and 23.

The inner pump electrode 22 is formed over the upper and lower solid electrolyte layers (the second solid electrolyte layer 6 and the first solid electrolyte layer 4) defining the first internal cavity 20, and the spacer layer 5 providing a side wall. Specifically, the ceiling electrode portion 22a is formed on the lower surface of the second solid electrolyte layer 6, providing a ceiling surface of the first internal cavity 20, a bottom electrode portion 22b is formed on the upper surface of the first solid electrolyte layer 4, providing a bottom surface, a side electrode portion (not shown) is formed on the side wall surface (inner surface) of the spacer layer 5, making both side wall portions of the first internal cavity 20, so as to connect those ceiling electrode portion 22a and the bottom electrode portion 22b, and the inner pump electrode 22 is disposed with a structure in a tunnel form at a portion where the side electrode portion is disposed.

The inner pump electrode 22 and the outer pump electrode 23 each are formed as a porous cermet electrode (for example, a cermet electrode of Pt and ZrO₂, having an Au content of 1 percent). The inner pump electrode 22 that contacts with a measurement-object gas is formed by using a material of which the reduction ability for NOx components in the measurement-object gas is lowered.

By passing a pump current Ip0 in a positive direction or a negative direction between the inner pump electrode 22 and the outer pump electrode 23 by applying a desired voltage Vp0 between the inner pump electrode 22 and the outer pump electrode 23, the main pump cell 21 is capable of pumping out oxygen in the first internal cavity 20 to the external space or pumping oxygen in the external space into the first internal cavity 20.

In order to detect an oxygen concentration (oxygen partial pressure) in an atmosphere in the first internal cavity 20, an electrochemical sensor cell, that is, a main pump control oxygen partial pressure detection sensor cell 80, is made up of the inner pump electrode 22, the second solid electrolyte layer 6, the spacer layer 5, the first solid electrolyte layer 4, the third substrate layer 3, and the reference electrode 42.

An oxygen concentration (oxygen partial pressure) in the first internal cavity 20 is found by measuring an electromotive force (voltage V0) in the main pump control oxygen partial pressure detection sensor cell 80. In addition, the pump current Ip0 is controlled by executing feedback control over the voltage Vp0 of a variable power source 24 such that the voltage V0 becomes a target value. With this configuration, it is possible to maintain the oxygen concentration in the first internal cavity 20 at a predetermined constant value.

The third diffusion control section 30 is a portion that applies predetermined diffusion resistance to a measurement-object gas of which the oxygen concentration (oxygen partial pressure) is controlled by operation of the main pump cell 21 in the first internal cavity 20 to guide the measurement-object gas to the second internal cavity 40. As illustrated in FIG. 1, the third diffusion control section 30 is provided between an upper surface of a third bridging portion Sc of the spacer layer 5 and the lower surface of the second solid electrolyte layer 6, and between a lower surface of the third bridging portion Sc and the upper surface of the first solid electrolyte layer 4. As illustrated in FIG. 3, the third bridging portion Sc is a portion that bridges to the left and right, and that located between the first internal cavity 20 and the second internal cavity 40, which are portions of the spacer layer 5 that has been punched out.

The second internal cavity 40 is provided as a space used to further adjust the oxygen partial pressure by using an auxiliary pump cell 50 for the measurement-object gas adjusted in the oxygen concentration (oxygen partial pressure) in the first internal cavity 20 in advance and then introduced through the third diffusion control section 30. With this configuration, it is possible to highly accurately maintain the oxygen concentration in the second internal cavity 40 at a constant value, so it is possible to measure a highly accurate NOx concentration with the gas sensor 100.

The auxiliary pump cell 50 is an auxiliary electrochemical pump cell made up of an auxiliary pump electrode 51 having a ceiling electrode portion 51a provided substantially all over the lower surface of the second solid electrolyte layer 6, facing the second internal cavity 40, the outer pump electrode 23 (not limited to the outer pump electrode 23, and an adequate electrode outside the sensor element 101 may be used), the second solid electrolyte layer 6, the spacer layer 5, and the first solid electrolyte layer 4.

The auxiliary pump electrode 51 is disposed in the second internal cavity 40 with a structure in a similar tunnel form to that of the inner pump electrode 22 provided in the above-described first internal cavity 20. In other words, the auxiliary pump electrode 51 has such a structure in a tunnel form that a ceiling electrode portion 51a is formed on the lower surface of the second solid electrolyte layer 6 providing the ceiling surface of the second internal cavity 40, a bottom electrode portion 51b is formed on the upper surface of the first solid electrolyte layer 4 providing the bottom surface of the second internal cavity 40, a side electrode portion (not shown) that couples those ceiling electrode portion 51a and bottom electrode portion 51b is formed on each of both wall surfaces of the spacer layer 5, providing a side wall of the second internal cavity 40. The auxiliary pump electrode 51, as well as the inner pump electrode 22, is formed by using a material of which the reduction ability for NOx components in the measurement-object gas is lowered.

By applying a desired voltage Vp1 between the auxiliary pump electrode 51 and the outer pump electrode 23, the auxiliary pump cell 50 is capable of pumping out oxygen in an atmosphere in the second internal cavity 40 to the external space or pumping oxygen from the external space into the second internal cavity 40.

In order to control an oxygen partial pressure in an atmosphere in the second internal cavity 40, an electrochemical sensor cell, that is, an auxiliary pump control oxygen partial pressure detection sensor cell 81, is made up of the auxiliary pump electrode 51, the reference electrode 42, the second solid electrolyte layer 6, the spacer layer 5, the first solid electrolyte layer 4, and the third substrate layer 3.

The auxiliary pump cell 50 performs pumping with a variable power source 52 of which the voltage is controlled in accordance with an electromotive force (voltage V1) detected by the auxiliary pump control oxygen partial pressure detection sensor cell 81. With this configuration, the oxygen partial pressure in an atmosphere in the second internal cavity 40 is controlled to a low partial pressure that substantially does not influence measurement of NOx.

Together with this, its pump current Ip1 is used to control the electromotive force of the main pump control oxygen partial pressure detection sensor cell 80. Specifically, the pump current Ip1 is input to the main pump control oxygen partial pressure detection sensor cell 80 as a control signal, and the gradient of the oxygen partial pressure in the measurement-object gas to be introduced from the third diffusion control section 30 into the second internal cavity 40 is controlled to be constantly unchanged by controlling the above-described target value of the voltage V0. When used as a NOx sensor, the oxygen concentration in the second internal cavity 40 is maintained at a constant value of about 0.001 ppm by the functions of the main pump cell 21 and auxiliary pump cell 50.

The fourth diffusion control section 60 is a portion that applies predetermined diffusion resistance to the measurement-object gas of which the oxygen concentration (oxygen partial pressure) is controlled by operation of the auxiliary pump cell 50 in the second internal cavity 40 to guide the measurement-object gas to the third internal cavity 61. The fourth diffusion control section 60 plays a role in limiting the amount of NOx flowing into the third internal cavity 61. As illustrated in FIG. 1, the fourth diffusion control section 60 is provided between an upper surface of a fourth bridging portion 5d of the spacer layer 5 and the lower surface of the second solid electrolyte layer 6, and between a lower surface of the fourth bridging portion 5d and the upper surface of the first solid electrolyte layer 4. As illustrated in FIG. 3, the fourth bridging portion 5d is a portion that bridges to the left and right, and that located between the second internal cavity 40 and the third internal cavity 61, which are portions of the spacer layer 5 that has been punched out.

The third internal cavity 61 is provided as a space used to perform a process related to measurement of a nitrogen oxide (NOx) concentration in a measurement-object gas on the measurement-object gas adjusted in oxygen concentration (oxygen partial pressure) in the second internal cavity 40 in advance and then introduced through the fourth diffusion control section 60. Measurement of a NOx concentration is mainly performed by operation of a measurement pump cell 41 in the third internal cavity 61.

The measurement pump cell 41 measures a NOx concentration in the measurement-object gas in the third internal cavity 61. The measurement pump cell 41 is an electrochemical pump cell made up of a measurement electrode 44 provided on the upper surface of the first solid electrolyte layer 4, facing the third internal cavity 61, the outer pump electrode 23, the second solid electrolyte layer 6, the spacer layer 5, and the first solid electrolyte layer 4. The measurement electrode 44 is a porous cermet electrode made of a material of which the reduction ability for NOx components in the measurement-object gas is raised as compared to the inner pump electrode 22. The measurement electrode 44 also functions as a NOx reduction catalyst that reduces NOx present in an atmosphere in the third internal cavity 61.

The measurement pump cell 41 is capable of pumping out oxygen produced as a result of decomposition of nitrogen oxides in an atmosphere around the measurement electrode 44 and detecting the amount of oxygen produced as a pump current Ip2.

In order to detect an oxygen partial pressure around the measurement electrode 44, an electrochemical sensor cell, that is, a measurement pump control oxygen partial pressure detection sensor cell 82, is made up of the first solid electrolyte layer 4, the third substrate layer 3, the measurement electrode 44, and the reference electrode 42. A variable power source 46 is controlled in accordance with an electromotive force (voltage V2) detected by the measurement pump control oxygen partial pressure detection sensor cell 82.

A measurement-object gas guided into the second internal cavity 40 reaches the measurement electrode 44 in the third internal cavity 61 through the fourth diffusion control section 60 in a situation in which the oxygen partial pressure is controlled. Nitrogen oxides in the measurement-object gas around the measurement electrode 44 are reduced (2NO→N₂+O₂) to produce oxygen. The produced oxygen is to be pumped by the measurement pump cell 41. At this time, the voltage Vp2 of the variable power source 46 is controlled such that the voltage V2 detected by the measurement pump control oxygen partial pressure detection sensor cell 82 is constant (target value). The amount of oxygen produced around the measurement electrode 44 is proportional to the concentration of nitrogen oxides in the measurement-object gas, so a nitrogen oxide concentration in the measurement-object gas is calculated by using the pump current Ip2 in the measurement pump cell 41.

When an oxygen partial pressure detection device is constructed as an electrochemical sensor cell by combining the measurement electrode 44, the first solid electrolyte layer 4, the third substrate layer 3, and the reference electrode 42, an electromotive force corresponding to the difference between the amount of oxygen produced by reduction of the NOx component in the atmosphere around the measurement electrode 44, and the amount of oxygen contained in the reference gas can be detected, and accordingly, the concentration of the NOx component in the measurement-object gas can be determined.

In addition, an electrochemical sensor cell 83 is made up of the second solid electrolyte layer 6, the spacer layer 5, the first solid electrolyte layer 4, the third substrate layer 3, the outer pump electrode 23, and the reference electrode 42, and it is possible to detect an oxygen partial pressure in a measurement-object gas outside the sensor by using an electromotive force (voltage Vref) obtained by the sensor cell 83.

In the gas sensor 100 having such a configuration, a measurement-object gas of which the oxygen partial pressure is maintained at a constantly unchanged low value (a value that substantially does not influence measurement of NOx) is supplied to the measurement pump cell 41 by operating the main pump cell 21 and the auxiliary pump cell 50. Therefore, it is possible to find a NOx concentration in the measurement-object gas in accordance with a pump current Ip2 that flows as a result of pumping out oxygen, produced by reduction of NOx, by the measurement pump cell 41 substantially in proportion to a NOx concentration in the measurement-object gas.

The sensor element 101 includes the heater portion 70 that plays a role in temperature adjustment for maintaining the temperature of the sensor element 101 by heating in order to increase the oxygen ion conductivity of the solid electrolyte. The heater portion 70 includes a heater connector electrode 71, a heater 72, a through-hole 73, a heater insulating layer 74, and a pressure release hole 75.

The heater connector electrode 71 is an electrode formed in such a manner as to be in contact with the lower surface of the first substrate layer 1. Connection of the heater connector electrode 71 to a heater power source 76 (see FIG. 2) allows electric power to be supplied from the heater power source 76 to the heater portion 70.

The heater 72 is an electric resistor formed in such a manner as to be sandwiched by the second substrate layer 2 and the third substrate layer 3 from upper and lower sides. The heater 72 is connected to the heater connector electrode 71 via the through-hole 73, and is supplied with electric power from a heater power source 76 to generate heat to increase and retain the temperature of the solid electrolyte forming the sensor element 101.

The heater 72 is embedded all over the region from the first internal cavity 20 to the third internal cavity 61, and is capable of adjusting the overall sensor element 101 to a temperature at which the solid electrolyte is activated.

The heater insulating layer 74 is an electrically insulating layer formed of an insulating material, such as alumina, on the top and lower surfaces of the heater 72. The heater insulating layer 74 is formed for the purpose of obtaining an electrical insulation property between the second substrate layer 2 and the heater 72 and an electrical insulation property between the third substrate layer 3 and the heater 72.

The pressure release hole 75 is a portion provided so as to extend through the third substrate layer 3 and the reference gas inlet layer 48 and communicate with the reference gas inlet space 43. The pressure release hole 75 is formed for the purpose of easing an increase in internal pressure resulting from an increase in temperature in the heater insulating layer 74.

As illustrated in FIG. 4, the control apparatus 95 includes the above-described variable power sources 24, 46, and 52, the above-described heater power source 76, and a controller 96. The controller 96 is a microprocessor including a CPU 97, a storage unit 98, and so forth. The storage unit 98 is an information-rewritable nonvolatile memory and is capable of storing, for example, various programs and various data. The controller 96 is configured to receive the voltage V0 of the main-pump-control oxygen-partial-pressure detection sensor cell 80, the voltage V1 of the auxiliary-pump-control oxygen-partial-pressure detection sensor cell 81, the voltage V2 of the measurement-pump-control oxygen-partial-pressure detection sensor cell 82, the voltage Vref of the sensor cell 83, the pump current Ip0 flowing through the main pump cell 21, the pump current Ip1 flowing through the auxiliary pump cell 50, and the pump current Ip2 flowing through the measurement pump cell 41. The controller 96 is configured to output control signals to the variable power sources 24, 46, and 52 and thus control the voltages Vp0, Vp1, and Vp2 that are to be output by the variable power sources 24, 46, and 52, thereby controlling the main pump cell 21, the measurement pump cell 41, and the auxiliary pump cell 50. The controller 96 is configured to output a control signal to the heater power source 76, thereby controlling the electric power to be supplied from the heater power source 76 to the heater 72. The storage unit 98 further stores target values V0*, V1*, V2*, and the like, to be described below. The CPU 97 of the controller 96 is configured to refer to the target values V0*, V1*, and V2* and thus control the pump cells 21, 41, and 50.

The controller 96 executes an auxiliary pump control process of controlling the auxiliary pump cell 50 so that the oxygen concentration in the second internal cavity 40 reaches a target concentration. Specifically, the controller 96 controls the auxiliary pump cell 50 by executing feedback control on the voltage Vp1 of the variable power source 52 so that the voltage V1 reaches a constant value (referred to as target value V1*). The target value V1* is defined as the value that causes the oxygen concentration in the second internal cavity 40 to reach a predetermined low oxygen concentration that does not substantially affect measurement of NOx.

The controller 96 executes a main pump control process of controlling the main pump cell 21 so that the pump current Ip1 flowing when the oxygen concentration in the second internal cavity 40 is adjusted by the auxiliary pump cell 50 in the auxiliary pump control process reaches a target current (referred to as target value Ip1*). Specifically, the controller 96 sets (feedback-controls) a target value (referred to as a target value V0*) of the voltage V0 based on the pump current Ip1 so that the pump current Ip1 caused to flow by the voltage Vp1 reaches the constant target current Ip1*. The controller 96 then performs feedback control on the voltage Vp0 of the variable power source 24 so that the voltage V0 reaches the target value V0* (in other words, the oxygen concentration in the first internal cavity 20 reaches the target concentration). The gradient of oxygen partial pressure in the measurement-object gas to be introduced from the third diffusion control section 30 into the second internal cavity 40 is made unchanged constantly by the main pump control process. The target value V0* is set to a value which causes the oxygen concentration in the first internal cavity 20 to be higher than 0% and reach a low oxygen concentration. The pump current Ip0 which flows during the main pump control process varies according to the oxygen concentration in the measurement-object gas (that is, the measurement-object gas in the vicinity of the sensor element 101) which flows into the measurement-object gas flow section through the gas inlet port 10. Thus, the controller 96 can also detect the oxygen concentration in the measurement-object gas based on the pump current Ip0.

The main pump control process and the auxiliary pump control process described above are also collectively referred as an adjustment pump control process. The first internal cavity 20 and the second internal cavity 40 are also collectively referred as an oxygen concentration adjustment chamber. The main pump cell 21 and the auxiliary pump cell 50 are also collectively referred as an adjustment pump cell. The controller 96 executes the adjustment pump control process, thus the adjustment pump cell adjusts the oxygen concentration in the oxygen concentration adjustment chamber.

In addition, the controller 96 executes a measurement pump control process of controlling the measurement pump cell 41 so that the voltage V2 reaches a constant value (referred to as a target value V2*) (in other words, so that the oxygen concentration in the third internal cavity 61 reaches a predetermined low concentration). Specifically, the controller 96 controls the measurement pump cell 41 by performing feedback control on the voltage Vp2 of the variable power source 46 so that the voltage V2 reaches the target value V2*. Oxygen is pumped out from the third internal cavity 61 by the measurement pump control process.

Execution of the measurement pump control process causes oxygen to be pumped out from the third internal cavity 61 so that the oxygen produced due to reduction of NOx in the measurement-object gas in the third internal cavity 61 become substantially zero. The controller 96 obtains a pump current Ip2 as a detected value corresponding to the oxygen produced in the third internal cavity 61 from the specific gas (in this case, NOx), and calculates the NOx concentration in the measurement-object gas based on the pump current Ip2.

The storage unit 98 stores a relational expression (for example, an expression of a linear function or a quadratic function) or a map as a correspondence relationship between the pump current Ip2 and the NOx concentration. Such a relational expression or map can be determined in advance by an experiment.

The controller 96 performs a heater control process of controlling the heater 72 by outputting a control signal to the heater power source 76 so that the temperature of the heater 72 reaches a target temperature (for example, 800° C.). Here, the temperature of the heater 72 can be expressed as a linear function of the resistance value of the heater 72. Thus, in the heater control process, the controller 96 calculates the resistance value of the heater 72 as a value (a value convertible to the temperature) regarded as the temperature of the heater 72, and performs feedback control on the heater power source 76 so that the calculated resistance value reaches a target resistance value (a resistance value corresponding to the target temperature). The controller 96 obtains, for example, the voltage of the heater 72 and the current flowing through the heater 72, and can calculate the resistance value of the heater 72 based on the obtained voltage and current. The controller 96 may calculate the resistance value of the heater 72, for example, by 3-terminal method or 4-terminal method. When passing an electric current through the heater 72, the heater power source 76 adjusts the electric power supplied to the heater 72 by changing the value of the voltage to be applied to the heater 72 based on, for example, a control signal from the controller 96.

The control apparatus 95 inclusive of the variable power sources 24, 46, and 52, the heater power source 76, and so forth illustrated in FIG. 4, is actually connected to each electrode inside the sensor element 101 via lead wires not illustrated, which are formed inside the sensor element 101, and the connector electrodes not illustrated (only the heater connector electrode 71 is shown in FIG. 1), which are formed on the rear end side of the sensor element 101.

Here, the first diffusion control section 11 will be explained in detail. The first diffusion control section 11 is provided in the upstream side of the first internal cavity 20, the second internal cavity 40, and the third internal cavity 61 of the measurement-object gas flow section. As described above, the first diffusion control section 11 has the diffusion control section 11a and the diffusion control section 11b. As illustrated in FIG. 2, the diffusion control section 11a has the first space portion 85a and a first porous body 87a, and the diffusion control section 11b has a second space portion 85b and a second porous body 87b. In FIG. 1, the first porous body 87a and the second porous body 87b are omitted from the drawing. The first and second space portions 85a and 85b and the first and second porous bodies 87a and 87b allow the measurement-object gas to flow in the front-rear direction (first direction). In the present embodiment, since the diffusion control section 11b (the second space portion 85b and the second porous body 87b) has the same configuration as the diffusion control section 11a (the first space portion 85a and the first porous body 87a), a detailed description of the diffusion control section 11b is omitted, and the diffusion control section 11a is described.

The first porous body 87a is provided so as to occupy a part of the cross-section of the diffusion control section 11a perpendicular to the front-rear direction. The first porous body 87a allows the measurement-object gas to pass therethrough. The first space portion 85a is a part of the cross-section perpendicular to the front-rear direction of the diffusion control section 11a which is not occupied by the first porous body 87a. The first porous body 87a is provided so as to occupy the lower half of the diffusion control section 11a, and the first space portion 85a is the upper half of the diffusion control section 11a. The diffusion control section 11a, the first space portion 85a, and the first porous body 87a each have a cross-section perpendicular to the front-rear direction that is approximately rectangular. The wording “rectangle” refers to a quadrilateral with all four angles being equal, and includes a square. In the present embodiment, the diffusion control section 11a, the first space portion 85a, and the first porous body 87a each have the shape of the cross-section perpendicular to the front-rear direction that has a length in the left-right direction that is greater than a length in the up-down direction. The first porous body 87a may be provided in the diffusion control section 11a so as to occupy 30% to 70% of the cross-section perpendicular to the front-rear direction of the diffusion control section 11a. In the present embodiment, as illustrated in FIG. 5, the first porous body 87a occupies 50% of the cross-section of the diffusion control section 11a. In the present embodiment, the first porous body 87a is provided from the front end to the rear end of the diffusion control section 11a. In addition, the first space portion 85a and the first porous body 87a each have the shape shown in FIG. 5 in the cross-section perpendicular to the front-rear direction at any portion of the diffusion control section 11a in the front-rear direction.

The first porous body 87a is formed of a ceramic porous body such as an alumina porous body, a zirconia porous body, a spinel porous body, a cordierite porous body, a titania porous body, and a magnesia porous body. In the present embodiment, the first porous body 87a is assumed to be formed of an alumina porous body.

In this way, since the diffusion control section 11a has the first space portion 85a and the first porous body 87a, it is possible to make a flow velocity of the measurement-object gas passing through the first porous body 87a different from a flow velocity of the measurement-object gas passing through the first space portion 85a in the cross-section of the diffusion control section 11a perpendicular to the front-rear direction. Specifically, the measurement-object gas passing through the first porous body 87a has a slower flow velocity than the measurement-object gas passing through the first space portion 85a. Thereby it is possible to cause turbulence in the measurement-object gas after passing through the first space portion 85a and the first porous body 87a. In the present embodiment, since the measurement-object gas that has passed through the first space portion 85a and the measurement-object gas that has passed through the first porous body 87a join in the buffer space 12 downstream of the diffusion control section 11a, it is possible to cause turbulence in the measurement-object gas in the buffer space 12. It is possible to increase a pressure loss of the measurement-object gas by the turbulence of the measurement-object gas. Similarly, it is possible to increase a pressure loss of the measurement-object gas by the diffusion control section 11b having the second space portion 85b and the second porous body 87b. These features make it possible to reduce the effect of the dynamic pressure of the measurement-object gas on the main pump cell 21 including the inner pump electrode 22 that is located downstream of the diffusion control section 11. For example, even if a composition of the measurement-object gas is the same, an operation of the main pump cell 21 (e.g. a value of the pump current Ip0 flowing through the main pump cell 21 and so forth) may differ if the dynamic pressure of the measurement-object gas differs. As a result, the dynamic pressure of the measurement-object gas may affect the accuracy in detection of the specific gas concentration. In contrast, in the first diffusion control section 11 of the present embodiment, the pressure loss of the measurement-object gas is increased by the first and second space portions 85a and 85b and the first and second porous bodies 87a and 87b, thereby reducing the effect of the dynamic pressure of the measurement-object gas on the main pump cell 21. In addition, since not only the main pump cell 21 but also the auxiliary pump cell 50 and the measurement pump cell 41 have auxiliary pump electrodes 51 and measurement electrodes 44 located downstream of the first diffusion control section 11, the effects on the auxiliary pump cell 50 and the measurement pump cell 41 of the dynamic pressure of the measurement-object gas can also be reduced.

It is sufficient if the first porous body 87a allows the measurement-object gas to pass therethrough, but a porosity of the first porous body 87a may be 10% or more, for example. The porosity of the first porous body 87a may be 15% or less. When the porosity of the first porous body 87a is 15% or less, the flow velocity of the measurement-object gas passing through the first porous body 87a can be made more different from the flow velocity of the measurement-object gas passing through the first space portion 85a, and the pressure loss of the measurement-object gas after passing through can be further increased. The same applies to a porosity of the second porous body 87b.

The first space portion 85a, the second space portion 85b, the first porous body 87a, and the second porous body 87b of the first diffusion control section 11 are located in the same cross-section perpendicular to the front-rear direction and form flow paths parallel to each other. In addition, the measurement-object gas that has passed through the diffusion control section 11a and the measurement-object gas that has passed through the diffusion control section 11b join in the buffer space 12 downstream of the first diffusion control section 11.

Now, an example of method for manufacturing the sensor element 101 included in the gas sensor 100 will be described. First, six non-calcinated ceramic green sheets each containing an oxygen-ion-conductive solid electrolyte, as a ceramic component are prepared. In each of these green sheets, a plurality of sheet holes to be used for positioning during printing or stacking as well as necessary through-holes and the like are provided in advance. Furthermore, the green sheet that is to become the spacer layer 5 is preliminarily subjected to a punching process or the like in which a space that is to become the measurement-object gas flow section is provided. More specifically, by punching out the green sheet that is to become the spacer layer 5 in such a way as to leave the parts that is to become the first to fourth bridging portions 5a to 5d, spaces that is to become the buffer space 12, the first internal cavity 20, the second internal cavity 40, and the third internal cavity 61 are formed. Likewise, the green sheet that is to become the first solid electrolyte layer 4 is subjected to a process for providing a space that is to become the reference-gas introduction space 43. Then, a pattern-printing process and a drying process for forming various patterns in the ceramic green sheets are performed in correspondence with the first substrate layer 1, the second substrate layer 2, the third substrate layer 3, the first solid electrolyte layer 4, the spacer layer 5, and the second solid electrolyte layer 6. Specific patterns to be formed include, for example, patterns that serve as electrodes such as the measurement electrode 44 described above, the lead wires connected to the electrodes, the connector electrodes such as the heater connector electrode 71, the reference-gas introduction layer 48, and the heater section 70. The pattern-printing process is performed in which a pattern-forming paste prepared in accordance with the properties required for an object to be formed is applied onto a green sheet by using a known screen printing technique. The drying process is performed by using a known drying technique. When the pattern-printing process and the drying process are completed, a printing process and a drying process are performed in which a bonding paste for stacking and bonding together the green sheets corresponding to the respective layers are printed and dried. Subsequently, a stacking process is performed in which the green sheets provided with the bonding paste are stacked in a predetermined order while being positioned at the sheet holes. Next, a pressure bonding process is performed in which the stacked green sheets are put under predetermined temperature and pressure conditions to be pressure bonded into a single layered body. The layered body thus obtained contains a plurality of sensor elements 101 therein. The layered body is cut into pieces each having the size of the sensor element 101. Then, a calcinating process is performed in which the layered body is calcinated at a predetermined calcination temperature, whereby a sensor element 101 is obtained.

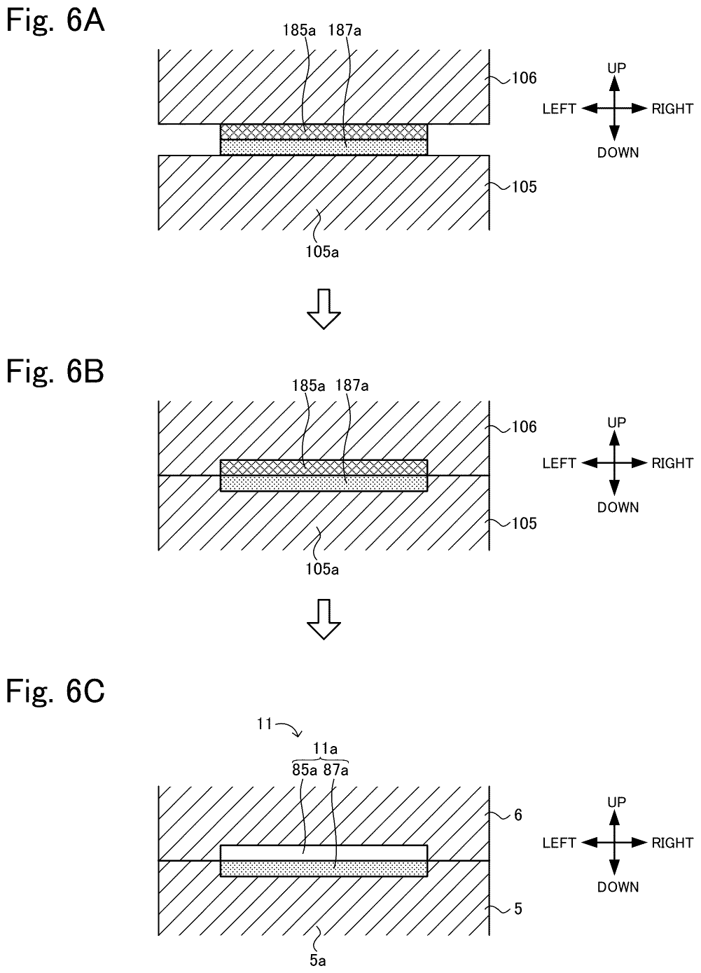

Here, a method of manufacturing the first diffusion control section 11 of the sensor element 101 will be described. FIGS. 6A to 6C are partial cross-sectional view showing a manufacturing process of the diffusion control section 11a. Since the diffusion control section 11b can be manufactured using the same method of manufacturing as the diffusion control section 11a, a detailed description of a method of manufacturing the diffusion control section 11b is omitted, and a method of manufacturing the diffusion control section 11a is described. First, as illustrated in FIG. 6A, in the pattern-printing process described above, a porous material layer 187a, which is to become the first porous body 87a, is printed and formed on the upper surface of a bridging portion 105a (corresponding to the first bridging portion 5a) of a green sheet 105, which is to become the spacer layer 5. The porous material layer 187a is formed using, for example, a paste containing the material of the first porous body 87a described above (in the present embodiment, alumina), a pore-forming material, a solvent, a binder, etc. The porosity of the first porous body 87a can be adjusted by adjusting the content ratio of the pore-forming material contained in the porous material layer 187a. Next, a vanishing material layer 185a is printed and formed on the upper surface of the porous material layer 187a. The vanishing material layer 185a is a layer for forming the first space portion 85a, and is formed using a paste containing a material that burns away in the calcinating process described above (e.g. theobromine and so forth). After printing is performed in this manner, the stacking process described above is performed, whereby a green sheet 106 which is to become the second solid electrolyte layer 6 is stacked on the vanishing material layer 185a as illustrated in FIG. 6A.

The layered body is manufactured by performing the pressure bonding process described above from the state shown in FIG. 6A, resulting the state shown in FIG. 6B. In the layered body, as illustrated in FIG. 6B, since the green sheet 105 and the green sheet 106 are softer than the porous material layer 187a and the vanishing material layer 185a, the upper surface of the green sheet 105 and the lower surface of the green sheet 106 are dented and the green sheet 105 and the green sheet 106 surround these layers from above, below, left and right. Viscosity (hardness) of the green sheets 105 and 106 can be adjusted, for example, by the kind or content ratio of a binder and a plasticizer contained in them. Viscosity (hardness) of the porous material layer 187a and the vanishing material layer 185a can be adjusted, for example, by the solid content ratio or the content ratio of the binder in the paste used for printing these layers. By adjusting the viscosity (hardness) of them, the green sheets 105 and 106 can be dented as illustrated in FIG. 6B.

After that, the layered body is cut into the pieces each having the size of the sensor element 101, and the calcinating process described above is performed on the cut layered body, whereby the vanishing material layer 185a burns and disappears, and becomes the first space portion 85a, as illustrated in FIG. 6C. The porous material layer 187a are calcinated and become the first porous body 87a. The green sheet 105, which includes the bridging portion 105a, and the green sheet 106 are calcinated and become the spacer layer 5, which includes the first bridging portion 5a, and the second solid electrolyte layer 6. In this way, the diffusion control section 11a with the first space portion 85a and the first porous body 87a is manufactured.

The correspondence relationships between the components in the present embodiment and the components in the present invention will now be clarified. Layers 1 to 6 according to the present embodiment each correspond to the solid electrolyte layer according to the present invention. The element body 102 corresponds to the element body. The first internal cavity 20, the second internal cavity 40, and the third internal cavity 61 each correspond to the internal cavity. The inner pump electrode 22, the auxiliary pump electrode 51, and the measurement electrode 44 each correspond to the inner electrode. The outer pump electrode 23 corresponds to the outer electrode. The main pump cell 21, the auxiliary pump cell 50, and the measurement pump cell 41 each correspond to the pump cell. The first diffusion control section 11 (the diffusion control section 11a and the diffusion control section 11b) corresponds to the diffusion control section. The first and second porous bodies 87a and 87b each correspond to the porous body. The first and second space portions 85a and 85b each correspond to the space portion.

In the sensor element 101 included in the gas sensor 100 according to the present embodiment described in detail above, the first diffusion control section 11 includes the first and second porous bodies 87a and 87b, and the first and second space portions 85a and 85b that are provided in at least a part of the first diffusion control section 11 in the front-rear direction (first direction). The first and second porous bodies 87a and 87b are provided so as to occupy the parts of the cross-section of the first diffusion control section 11 perpendicular to the front-rear direction and allow the measurement-object gas to pass therethrough. The first and second space portions 85a and 85b include the parts of the cross-section not occupied by the first and second porous bodies 87a or 87b and allow the measurement-object gas to pass therethrough. As a result, it is possible to make the flow velocities of the measurement-object gas passing through the first and second porous bodies 87a and 87b different from the flow velocities of the measurement-object gas passing through the first and second space portions 85a and 85b in the cross-section of the first diffusion control section 11 perpendicular to the front-rear direction. Thereby it is possible to cause turbulence in the measurement-object gas after passage and increase the pressure loss of the measurement-object gas. Therefore, the effects of the dynamic pressure of measurement-object gas on the main pump cell 21, auxiliary pump cell 50, and measurement pump cell 41 that is located downstream of the first diffusion control section 11 can be reduced.

In addition, since the porosity of the first porous body 87a is 15% or less, the flow velocity of the measurement-object gas passing through the first porous body 87a can be made more different from the flow velocities of the measurement-object gas passing through the first and second space portions 85a and 85b, and the pressure loss of the measurement-object gas after passing through can be increased. The same advantageous effect can be obtained when the porosity of the second porous body 87b is 15% or less.

Note that the present invention is not limited to the above-described embodiment at all, and may be, of course, implemented in various modes within the technical scope of the present invention.

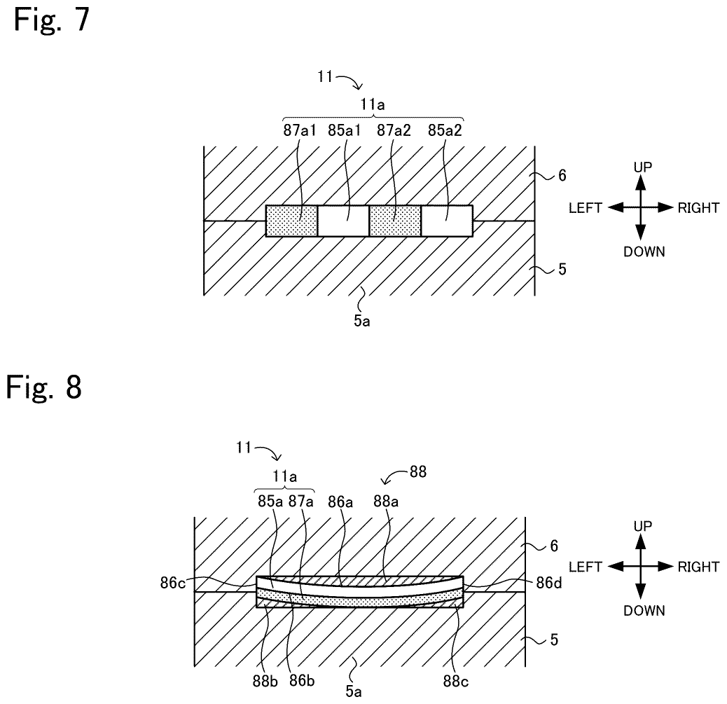

For example, in the embodiment described above, the diffusion control section 11a has one first space portion 85a and one first porous body 87a, but at least one of the space portion and the porous body may be provided in a plurality. For example, as illustrated in FIG. 7, the diffusion control section 11a may have space portions 85al and 85a2 and porous bodies 87a1 and 87a2 arranged alternately in the left-right direction. A plurality of the space portions 85a1 and 85a2 are located in the same cross-section perpendicular to the front-rear direction and form flow paths parallel to each other. A plurality of the porous bodies 87a1 and 87a2 are located in the same cross-section perpendicular to the front-rear direction and form flow paths parallel to each other.

In the above embodiment, the first space portion 85a, and the first porous body 87a each have a cross-section perpendicular to the front-rear direction that is approximately rectangular, but the present invention is not limited to this. For example, an embodiment illustrated in FIG. 8 may be adopted. In FIG. 8, the first space portion 85a and the first porous body 87a each have a cross-section perpendicular to the front-rear direction that has a shape of a rectangle with curved long sides. Specifically, in a cross-section perpendicular to the front-rear direction, the first space portion 85a has the first to fourth portions 86a to 86d as an outer circumference of the cross-section. The first portion 86a, the second portion 86b, the third portion 86c, and the fourth portion 86d correspond to the upper, lower, left, and right sides of the outer circumference of the cross-section of the first space portion 85a. The first portion 86a and the second portion 86b face each other in the up-down direction (the layered direction) and extend along the left-right direction (the second direction), respectively. The third portion 86c and the fourth portion 86d face each other in the left-right direction and extend along the up-down direction, respectively. The first portion 86a and the second portion 86b each have a curved shape. The first portion 86a and the second portion 86b are each curved to form a convex shape on the same side in the up-down direction (in this case, lower side). The third portion 86c and the fourth portion 86d each have a straight line shape along the up-down direction. The cross-section of the first space portion 85a has a length in the left-right direction that is greater than a length in the up-down direction. The first porous body 87a also is shaped similarly to the first space portion 85a. In addition, the entire of the diffusion control section 11a also has a cross-section perpendicular to the front-rear direction that has a shape of a rectangle with curved long sides, similar to the first space portion 85a and the first porous body 87a.

In the embodiment illustrated in FIG. 8 the element body 102 has a structural portion 88 that constitutes at least a part of the outer periphery of the diffusion control section 11a (an outer wall of the diffusion control section 11a). The structural portion 88 has a first structural portion 88a, a second structural portion 88b, and a third structural portion 88c. The first structural portion 88a is provided on the lower surface of the second solid electrolyte layer 6. The first structural portion 88a is thin at both left and right ends in the cross-section perpendicular to the front-rear direction and becomes thicker toward a center in the left-right direction. With this configuration, a lower surface of the first structural portion 88a thus curves so that the lower surface has a convex shape to the down direction, thereby constituting an upper side of the diffusion control section 11a, that is the first portion 86a of the first space portion 85a. The second structural portion 88b and the third structural portion 88c are provided on the upper surface of the spacer layer 5. The second structural portion 88b is thick at left end in the cross-section perpendicular to the front-rear direction and becomes thinner toward a center of the first space portion 85a in the left-right direction. The third structural portion 88c is thick at right end in the cross-section perpendicular to the front-rear direction and becomes thinner toward a center of the first space portion 85a in the left-right direction. With these configurations, an upper surface of the second structural portion 88b and an upper surface of the third structural portion 88c as a whole are curved so that the upper surfaces have a convex shape to the down direction, thereby forming a shape of a lower side of the diffusion control section 11a, that is a shape of a lower side of the first porous body 87a. Left and right sides of the diffusion control section 11a (including the third portion 86c and the fourth portion 86d of the first space portion 85a) that has the straight line shape are constituted by not the structural portion 88 but the spacer layer 5 and second solid electrolyte layer 6, respectively. The structural portion 88 is made of a dense material that does not allow the measurement-object gas to flow through, and in the present embodiment is made of alumina ceramics. The first space portion 85a, the first porous body 87a, and the structural portion 88 each have the same shape (the shape shown in FIG. 8) in the cross-section perpendicular to the front-rear direction at any portion in the front-rear direction.

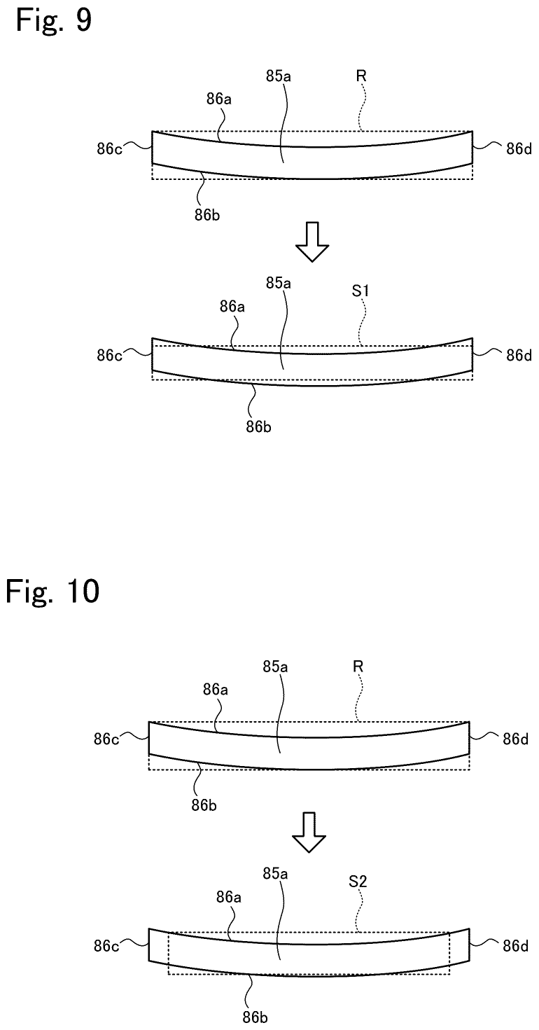

In FIG. 8, The cross-section of the first space portion 85a perpendicular to the front-rear direction has a predetermined shape whose outer circumference is longer than that of a reference rectangle. The reference rectangle will be described using FIG. 9. First, it is considered that a circumscribed rectangle R of the cross-section of the first space portion 85a, which is composed of two sides parallel to the up-down direction and two sides parallel to the left-right direction (upper part of FIG. 9). Next, the circumscribed rectangle R is modified by shortening the short sides while maintaining the length of the long sides so that the area is the same as that of the cross-section of the first space portion 85a, and the resulting rectangle is defined as a reference rectangle S1 (lower part of FIG. 9). If the circumscribed rectangle R is a square, it is sufficient to shorten either two sides of the two sides parallel to the up-down direction or the two sides parallel to the left-right direction within the circumscribing rectangle R. The reference rectangle S1 has a shape corresponding to the conventional cross-sectional shape (rectangle) of the space of the diffusion-controlling portion 11a, and is a rectangle with the same cross-sectional area as the first space portion 85a so that it can be compared with the first space portion 85a. The cross-sectional area of the first space portion 85a is the same as the reference rectangle S1, and, unlike the reference rectangle S1, the first portion 86a and the second portion 86b each have the curved shape, so the length of the outer circumference of the first space portion 85a is longer than the reference rectangle S1. Furthermore, the greater the degree of curvature of the first portion 86a and second portion 86b, the longer the length of the outer circumference of the first space portion 85a can be made. Since the shape of the cross-section of the first space portion 85a is the predetermined shape with a longer outer circumference than a reference rectangle, a hydraulic diameter of the first space portion 85a is smaller than a hydraulic diameter of the reference rectangle S1. Here, the hydraulic diameter is expressed as four times the value obtained by dividing the cross-sectional area by the wetted perimeter (i.e., the length of the outer circumference of the cross-section) (the hydraulic diameter=cross-sectional area/wetted perimeter×4). Since the cross-sectional area of the first space portion 85a is equal to that of the reference rectangle S1, if the length of the outer circumference of the first space portion 85a (the sum of the lengths of the first to fourth portions 86a to 86d) is longer than the length of the outer circumference of the reference rectangle S1, the hydraulic diameter of the first space portion 85a will be smaller than that of the reference rectangle S1. Furthermore, the small hydraulic diameter increases the pressure loss of the measurement-object gas passing through the first space portion 85a. Therefore, in the diffusion control section 11a of FIG. 8, the diffusion control section 11a not only has a first space portion 85a and a first porous body 87a, but also has the shape of the cross-section perpendicular to the front-rear direction of the first space portion 85a that is the predetermined shape, so that the effect of the dynamic pressure of the measurement-object gas on the main pump cell 21, the auxiliary pump cell 50, and the measuring pump cell 41 that is located downstream of the diffusion control section 11a can be reduced. In the embodiment in FIG. 8, the first porous body 87a and the entire of the diffusion control section 11a also each have a cross-section perpendicular to the front-rear direction that has the predetermined shape, similar to the first space portion 85a.

In addition, it is also possible to increase the pressure loss by reducing the cross-sectional area of the first space portion 85a. For example, even if the shape of the cross-section of the first space 85a is made smaller than the cross-sectional area while remaining the same rectangular shape as the reference rectangle S1, the pressure loss will be higher than that of the reference rectangle S1. However, if the cross-sectional area is reduced, the flow rate of the measurement-object gas passing through the first space portion 85a itself will decrease, and for example, the pump currents Ip0, Ip1, and Ip2 flowing through the main pump cell 21, auxiliary pump cell 50, and measurement pump cell 41 downstream of the first diffusion control section 11 will always be in the range of small values, and the accuracy in detection of the specific gas concentration by the gas sensor 100 may decrease. In contrast, in the diffusion control section 11a shown in FIG. 8, the cross-sectional area of the first space portion 85a is a predetermined shape, so it is possible to increase the pressure loss without reducing the cross-sectional area of the first space portion 85a compared to conventional shapes such as the reference rectangle S1. Therefore, it is possible to reduce the effects that the main pump cell 21 and so forth receive from the dynamic pressure of the measurement-object gas while maintaining the flow rate of the measurement-object gas passing through the diffusion control section 11a.

In addition, a definition different from that of the reference rectangle S1 illustrated in FIG. 9 may be used as the definition of the reference rectangle. For example, as illustrated in FIG. 10, the reference rectangle S2 (lower part of FIG. 10) may be a rectangle that is obtained so as to deform the circumscribed rectangle R is the same as the cross-sectional area of the first space portion 85a (upper part of FIG. 10). Alternatively, the circumscribing rectangle R may be a reference rectangle S3 (not shown in the figure) in which the length of each side has been changed so that the area is the same as the cross-sectional area of the first space portion 85a and the overlapping area with the cross-sectional area of the first space portion 85a is maximized. Note that the reference rectangle S3 may end up having the same shape as the reference rectangle S1 or the reference rectangle S2. If the shape of the cross-section perpendicular to the front-rear direction of the first space portion 85a is a predetermined shape with a longer outer circumference than at least one of the reference rectangles S1 to S3 defined in this way, the hydraulic diameter of the first space portion 85a will be smaller than that of the reference rectangle, and it can be said that the first space portion 85a has a shape that causes a higher pressure loss for the measurement-object gas passing through it. In the first space portion 85a shown in FIG. 8, the degree of curvature of the first portion 86a and second portion 86b is determined so that the cross-sectional shape of the first space portion 85a has a predetermined shape with a longer outer circumference than any of the reference rectangles S1 to S3.

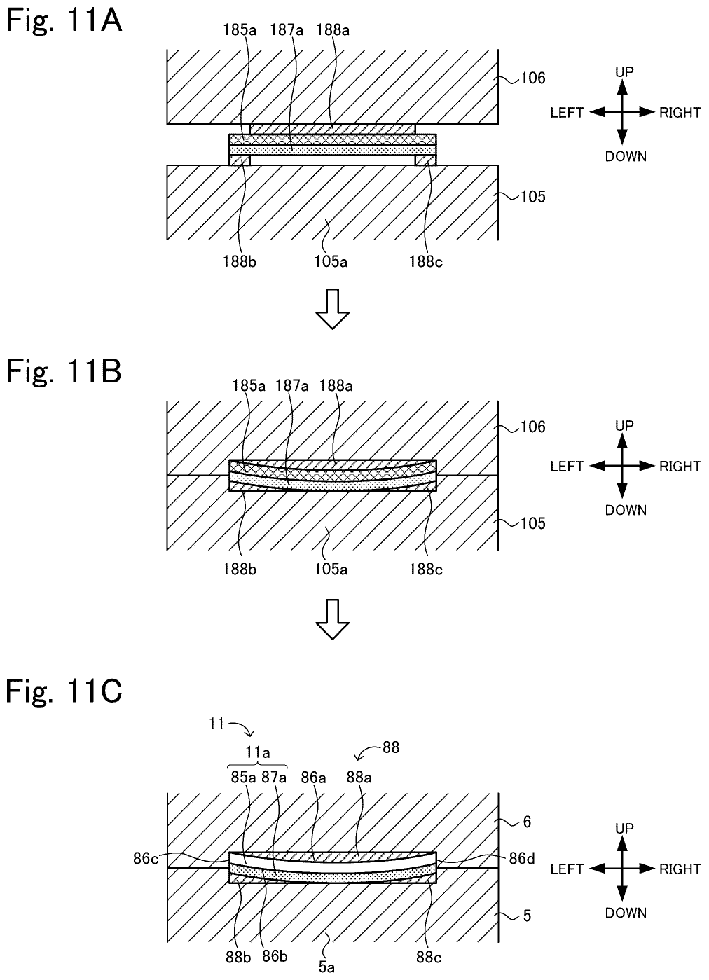

A method of manufacturing the diffusion control section 11a in FIG. 8 will be described with reference to FIGS. 11A to 11C. First, as illustrated in FIG. 11A, in the pattern-printing process described above, a second structural material layer 188b and a third structural material layer 188c, which is to become the second structural portion 88b and the third structural portion 88c respectively, are printed and formed on the upper surface of a bridging portion 105a (corresponding to the first bridging portion 5a) of a green sheet 105, which is to become the spacer layer 5. The second structural material layer 188b and the third structural material layer 188c are formed using a paste that contains alumina for example, as a ceramic component. The second structural material layer 188b and the third structural material layer 188c may also be a paste of the same material as the porous material layer 187a, except that the paste does not contain a pore-forming material. The second structural material layer 188b and the third structural material layer 188c are formed that the second structural material layer 188b and the third structural material layer 188c tend to become thinner toward a center in the left-right direction as a whole, so that their upper surfaces will have a shape that corresponds to the curved shape of the lower side of the first porous body 87a and the second portion 86b of the first space portion 85a after the pressure bonding process. In FIG. 11A, the second structural material layer 188b and the third structural material layer 188c are formed at the positions corresponding to the left and right ends of the diffusion control section 11a, respectively, on the upper surface of the first bridging portion 5a, and no structural material layer is formed in the area around the center of the diffusion control section 11a in the left-right direction. Next, a porous material layer 187a is printed and formed on the upper surface of the green sheet 105, in a region from a left edge of the second structural material layer 188b to a right edge of the third structural material layer 188c, and a vanishing material layer 185a is formed on the upper surface of the porous material layer 187a. Then, a first structural material layer 188a, which is to become the first structural portion 88a, is printed and formed on an upper surface of the vanishing material layer 185a. The first structural material layer 188a is formed using a paste that contains alumina as a ceramic component, in the same way as the second structural material layer 188b and the third structural material layer 188c. The first structural material layer 188a is formed so that the first structural material layer 188a tends to become thicker toward a center in the left-right direction, so that a lower surface of the first structural material layer 188a will have a shape that corresponds to the curved shape of the first portion 86a of the first space portion 85a after the pressure bonding process. In FIG. 11A, a width of the first structural material layer 188a in the left-right direction is made smaller than a width of the vanishing material layer 185a in the left-right direction, and the first structural material layer 188a is not formed in positions corresponding to the left and right ends of the first space portion 85a. After printing is performed in this manner, the stacking process described above is performed, whereby a green sheet 106 which is to become the second solid electrolyte layer 6 is stacked on the first structural material layer 188a as illustrated in FIG. 11A.

The layered body is manufactured by performing the pressure bonding process described above from the state shown in FIG. 11A, resulting the state shown in FIG. 11B. In the layered body, as illustrated in FIG. 11B, since the green sheet 105 and the green sheet 106 are softer than the vanishing material layer 185a, the porous material layer 187a, and the first to third structural material layers 188a to 188c, the upper surface of the green sheet 105 and the lower surface of the green sheet 106 are dented and the green sheet 105 and the green sheet 106 surround these layers from above, below, left and right. In addition, the vanishing material layer 185a, the porous material layer 187a, and the first to third structural material layers 188a to 188c are deformed by being pressed from above and below by the green sheets 105 and 106, whereby the vanishing material layer 185a takes the shape of the first space portion 85a, the porous material layer 187a takes the shape of the first porous body 87a, and the first to third structural material layers 188a to 188c take the shapes of the first to third structural portions 88a to 88c. In addition, the viscosity of the first to third structural material layers 188a to 188c can be adjusted using the same method as for the porous material layer 187a and the vanishing material layer 185a.

After that, the layered body is cut into the pieces each having the size of the sensor element 101, and the calcinating process described above is performed on the cut layered body, whereby the vanishing material layer 185a burns and disappears, and becomes the first space portion 85a, as illustrated in FIG. 11C. In addition, the green sheet 105, which includes the bridging portion 105a, and the green sheet 106 are calcinated and become the spacer layer 5, which includes the first bridging portion 5a, and the second solid electrolyte layer 6. The porous material layer 187a are calcinated and become the first porous body 87a. The first to third structural material layers 188a to 188c are calcinated and become the first to third structural portions 88a to 88c. In this way, the diffusion control section 11a with the first space portion 85a and the first porous body 87a is manufactured. A diffusion control section 11a having a first space portion 85a and a first porous body 87a with the cross-section perpendicular to the front-rear direction that has the predetermined shape is formed.

In FIG. 8, the embodiment described above, the first portion 86a and the second portion 86b of the first space portion 85a each have the curved shape, but when the cross-sectional shape of the first space portion 85a is the predetermined shape described above (the shape with a longer outer circumference than at least one of the reference rectangles S1 to S3), the cross-sectional shape of the first space 85a can be of various embodiments.

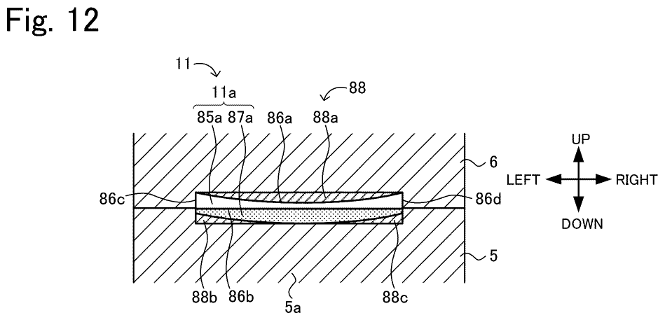

For example, one of the first portion 86a and the second portion 86b may have a curved shape and the other may have a straight shape. In a diffusion control section 11a according to a modification illustrated in FIG. 12, a first part 86a has a curved shape and the second part 86b has a straight shape. In FIG. 12, a first porous body 87a has a straight upper side and a curved lower side. The diffusion control section 11a in FIG. 12 can be formed, for example, by forming the vanishing material layer 185a so that its thickness tends to become thinner toward a center in the left-right direction, and forming the porous material layer 187a so that its thickness tends to become thicker toward a center in the left-right direction, in FIG. 11A. For example, by forming the vanishing material layer 185a by printing multiple times and forming the center portion of the vanishing material layer 185a in the left-right direction with fewer printing times than the other portions, the thickness of the vanishing material layer 185a can be made partially different so that the thickness at the center portion in the left-right direction is thinner.

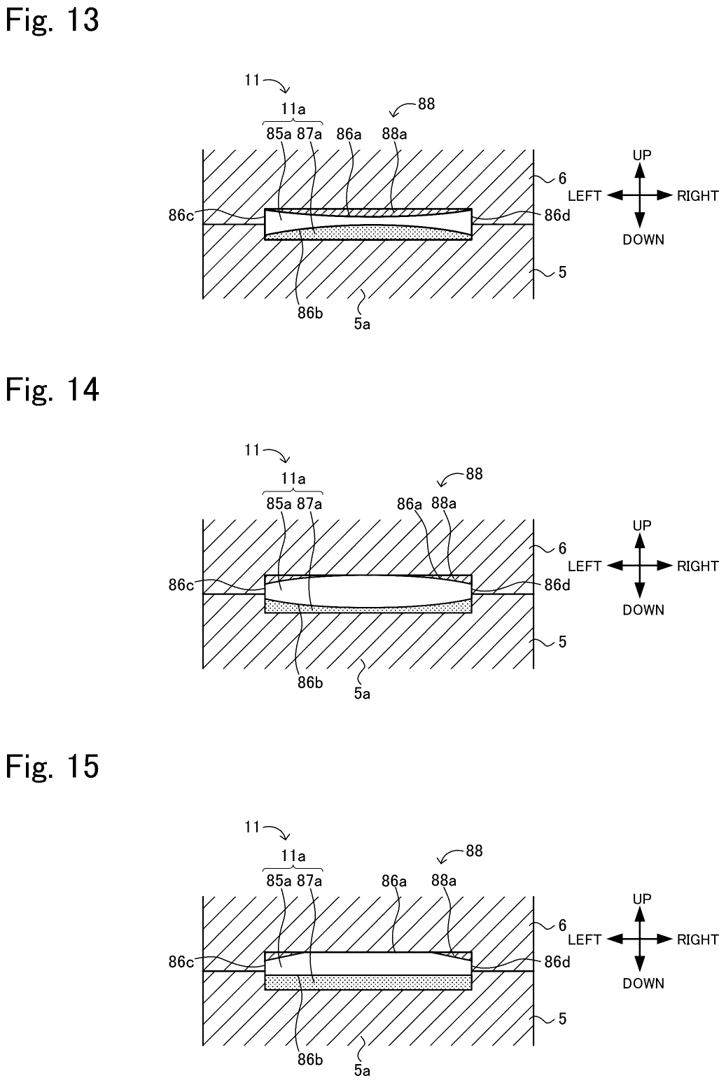

In FIG. 8, the overall shape of the first portion 86a of the first space portion 85a is the curved shape in the embodiment described above, but at least a part of the first portion 86a may have a curved shape, such as a combination of straight and curved shapes. The same applies to the second portion 86b. Although the first portion 86a and the second portion 86b in FIG. 8 each curve so that they each have the convex shape to the down direction, at least one of them may curve so that it is a convex shape to the up direction. Although the first portion 86a and the second portion 86b in FIG. 8 each have a convex shape on the same side in the up-down direction, the present invention is not limited to this embodiment, and for example, as shown in FIG. 13, the first portion 86a and the second portion 86b may each have a convex shape toward a side where they approach each other in the up-down direction. As shown in FIG. 14, the first portion 86a and the second portion 86b may each have a convex shape toward a side where they are separated from each other in the up-down direction. At least a part of the first portion 86a and/or at least a part of the second portion 86b may each have a polygonal line shape rather than a curved shape. For example, as illustrated in FIG. 15, the first portion 86a may be formed into a polygonal line shape having two bending portions, and the second portion 86b may be formed into a straight line shape. In these embodiments cases also, since the cross-section perpendicular to the front-rear direction of the first space portion 85a has the predetermined shape described above (the shape with a longer outer circumference than at least one of the reference rectangles S1 to S3), the effect of the dynamic pressure of the measurement-object gas on the main pump cell 21 and so forth that is located downstream of the diffusion control section 11a can be further reduced. In FIG. 15, the shape of the cross-section perpendicular to the front-rear direction of the first space portion 85a is formed into a polygonal shape (here, a hexagonal shape) having five or more sides.

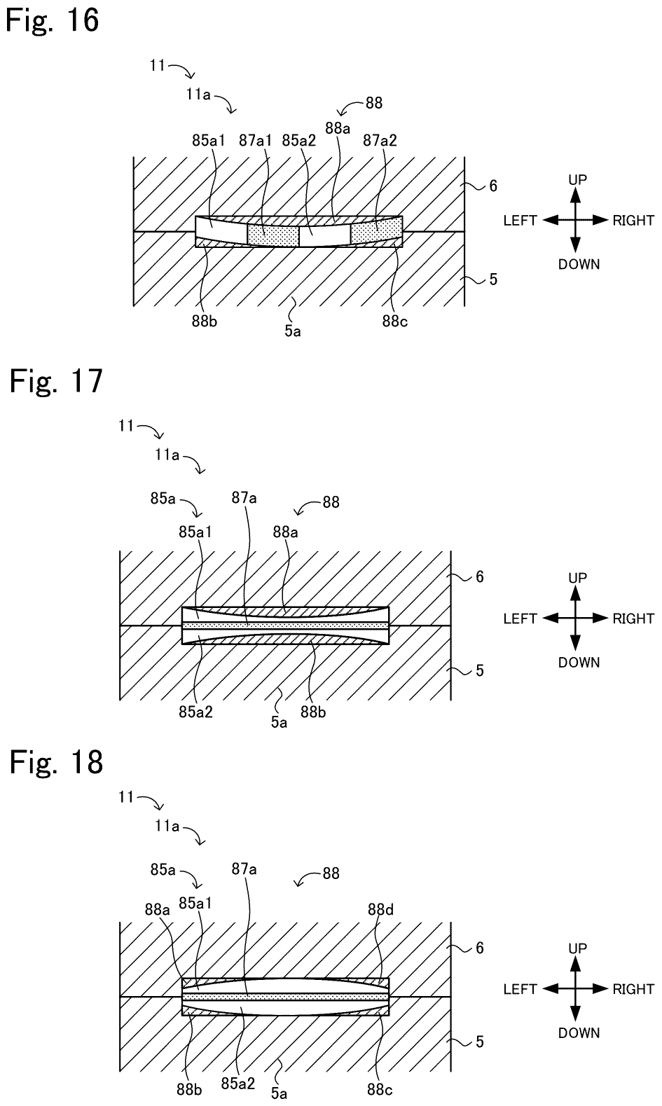

The embodiment illustrated in FIG. 16 may be adopted. In the embodiment of FIG. 16, the sensor element 101 includes a structural portion 88 similar to that of FIG. 12, and the diffusion control section 11a includes space portions 85a1 and 85a2 arranged alternately in the left-right direction and porous bodies 87a1 and 87a2 same as in FIG. 7. In the diffusion control section 11a of FIG. 16, each of the space portions 85a1 and 85a2 has a curved shape such that an upper side (first portion) and a lower side (second portion) thereof each have a convex shape in the down direction, and has a cross-section perpendicular to the front-rear direction that has the predetermined shape described above. In addition, in the embodiment shown in FIG. 16, the porous bodies 87a1 and 87a2 and the entire of the diffusion control section 11a also have a cross-section perpendicular to the front-rear direction that has the predetermined shape, similar to the space portions 85al and 85a2.

The first space portion 85a described above may be divided into a plurality of space portions by the first porous body 87a. For example, as illustrated in FIGS. 17 and 18, the first space portion 85a may be divided into two space portions, an upper space portion 85a1 and a lower space portion 85a2, by the first porous body 87a. In FIG. 17, the space portion 85a1 has a curved shape such that the upper side (first portion) thereof has a convex shape toward the down direction, as in the first space portion 85a in FIG. 12, and has a cross-section perpendicular to the front-rear direction that has the predetermined shape described above. The upper side of the space portion 85al is constituted by the lower surface of the first structural portion 88a. The lower side (second part) and left and right sides of the space portion 85a1 each have a straight shape. The lower side of the space portion 85a1 is constituted by the upper surface of the first porous body 87a. The space portion 85a2 has a curved shape such that the lower side (second portion) thereof has a convex shape toward the up direction, and has a cross-section perpendicular to the front-rear direction that has the predetermined shape described above. The lower side of the space portion 85a2 is constituted by the upper surface of the second structural portion 88b. The upper side (first portion) and the left and right sides of the space portion 85a2 each have a straight portion. In FIG. 18, the space portion 85a1 has a curved shape such that the upper side (first portion) thereof has a convex shape toward the up direction, and has a cross-section perpendicular to the front-rear direction that has the predetermined shape described above. The upper side of the space portion 85al is constituted by the lower surfaces of the first structural portion 88a and the fourth structural portion 88d. The space portion 85a2 has a curved shape such that the lower side (second portion) thereof has a convex shape toward the down direction, and has a cross-section perpendicular to the front-rear direction that has the predetermined shape described above. The lower side of the space portion 85a2 is constituted by the upper surfaces of the second structural portion 88b and the third structural portion 88c. The diffusion control section 11a in FIG. 18 has the same configuration as the diffusion control section 11a in FIG. 17, except for the above-mentioned points. The embodiments of FIGS. 7 and 16 described above can also be said to be examples of the embodiment in which the first space portion 85a is divided into a plurality of space portions by the first porous body 87a.

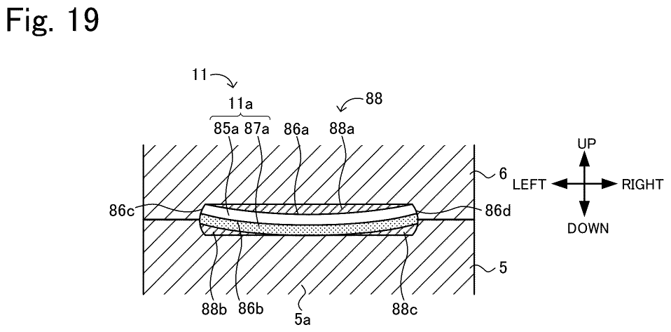

In FIG. 8, the third portion 86c and the fourth portion 86d of the first space portion 85a each have the straight line shape along the up-down direction in the cross-section perpendicular to the front-rear direction, but the present invention is not limited to this embodiment. For example, a third portion 86c and a fourth portion 86d may each have a curved shape as illustrated in FIG. 19. In FIG. 19, the third portion 86c and the fourth portion 86d each have a convex shape toward a side where they are separated from each other in the left-right direction. For example, by adjusting the viscosity of the green sheets 105 and 106, the vanishing material layer 185a, the porous material layer 187a, and the first to third structural material layers 188a to 188c shown in FIG. 11A, the vanishing material layer 185a, the porous material layer 187a, and the first to third structural layers 188a to 188c can be deformed so as to bulge to the left and right during the pressure bonding process, and the first space portion 85a having the shapes of the third portion 86c and the fourth portion 86d illustrated in FIG. 19 can be formed.

When the cross-sectional shape of the first space portion 85a is the predetermined shape described above (the shape with a longer outer circumference than at least one of the reference rectangles S1 to S3), in the modifications described above, the first space portion 85a has the first to fourth portions 86a to 86d, but the present invention is not limited to this embodiment. For example, in FIG. 14, the first space portion 85a does not have the third portion 86c and the fourth portion 86d, and the shape of the outer circumference of the cross-section of the first space portion 85a may be a shape in which the first portion 86a and the second portion 86b are directly connected at the left and right ends.

In the embodiment described above, the diffusion control section 11a has the same shape (the shape shown in FIG. 5) in the cross-section perpendicular to the front-rear direction at any portion in the front-rear direction, but the diffusion control section 11a is not limited to this embodiment, and it is sufficient if a first space portion 85a and a first porous body 87a exist in a cross-section in at least a part of the diffusion control section 11a in the front-rear direction. For example, only a front end (upstream end) portion of the diffusion control section 11a may have the cross-sectional shape illustrated in FIG. 5, and the other portions may have a cross-section perpendicular to the front-rear direction without the first porous body 87a, and the entire of the cross-section may be a space portion. Similarly, when the cross-section of the first space portion 85a and the space portions 85a1 and 85a2 of the diffusion control section 11a is formed into the predetermined shape described above as shown in FIG. 8 and FIGS. 12 to 19, it is sufficient that the cross-section in at least a part of the front-rear direction of them is formed into the predetermined shape.

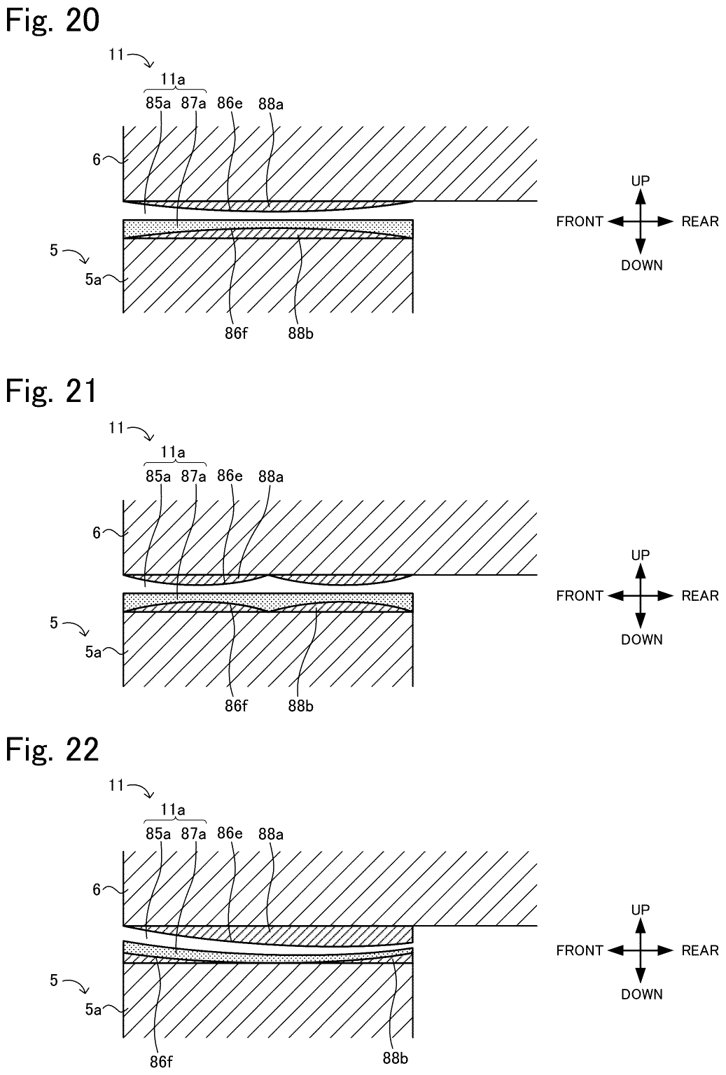

In addition, the diffusion control section 11a may be formed such that a cross-sectional area of the cross-section perpendicular to the front-rear direction is smaller in at least a part of a downstream side of the upstream end than in the upstream end. For example, a configuration shown in FIG. 20 may be adopted. FIG. 20 shows a cross-section of the diffusion control section 11a parallel to the front-rear direction. In FIG. 20, the upper and lower outlines 86e and 86f of the cross-section of the diffusion control section 11a parallel to the front-rear direction each have a curved shape, and the outlines 86e and 86f both extend so as to curve with respect to the front-rear direction. In addition, the outlines 86e and 86f each have a convex shape toward a side where they approach each other in the up-down direction. As a result, the cross-sectional area of the cross-section of the diffusion control section 11a perpendicular to the front-rear direction is the smallest at a center in the front-rear direction. In this way, the pressure loss of the measurement-object gas passing through a portion of the diffusion control section 11a having a smaller cross-sectional area than the upstream end (for example, a central portion in the front-rear direction) increases. Therefore, by this shape of this diffusion control section 11a, the effect of the dynamic pressure of measurement-object gas on the main pump cell 21, auxiliary pump cell 50, and measurement pump cell 41 that are located downstream of the first diffusion control section 11 can be also reduced. The shapes of the outlines 86e and 86f of the diffusion control section 11a in FIG. 20 can be adjusted by the shapes of the first to third structural portions 88a to 88c, similarly to the shapes of the first portion 86a and the second portion 86b. In FIG. 20, not only the entire of the diffusion control section 11a but also each of the first space portion 85a and the first porous body 87a are formed such that a cross-sectional area of the cross-section perpendicular to the front-rear direction is smaller in at least a part of a downstream side of the upstream end than in the upstream end.

Instead of the embodiment in FIG. 20, embodiments in FIG. 21 or 22 may be adopted. In FIG. 21, an outline 86e has a curved shape formed by connecting two outlines 86e of FIG. 20 front to back, and an outline 86f has a curved shape formed by connecting two outlines 86f of FIG. 20 front to back. Therefore, the outlines 86e and 86f in FIG. 21 each have two sets of convex shapes toward a side where they approach each other in the up-down direction. As a result, in FIG. 21, there are two locations where the cross-sectional area of the cross-section of the diffusion control section 11a perpendicular to the front-rear direction is the smallest, and these two locations are located in the middle of the front-rear direction (at positions other than the front end and rear end), and the pressure loss of the measurement-object gas passing through these two locations increases. In FIG. 22, the upper and lower outlines 86e and 86f of the cross-section of the diffusion control section 11a parallel to the front-rear direction each have a curved shape, and the outlines 86e and 86f both extend so as to curve with respect to the front-rear direction. In addition, the outlines 86e and 86f each have a convex shape on the same side (lower side) in the up-down direction. Furthermore, the outlines 86e and 86f are shaped such that the distance between them in the up-down direction becomes smaller as they approach the rear end relative to the front end. As a result, the diffusion control section 11a in FIG. 22 has a cross-sectional area of the cross-section perpendicular to the front-rear direction that is the smallest at the rear end. In this way, in FIG. 22, the pressure loss of the measurement-object gas passing through the rear end of the diffusion control section 11a increases. In FIGS. 21 and 22, not only the entire of the diffusion control section 11a but also each of the first space portion 85a and the first porous body 87a are formed such that a cross-sectional area of the cross-section perpendicular to the front-rear direction is smaller in at least a part of a downstream side of the upstream end than in the upstream end.

The diffusion control section 11a illustrated in FIGS. 20 to 22 has a first space portion 85a and a first porous body 87a in a cross-section perpendicular to the front-rear direction in any part of the front-rear direction, but there may be a part in which the first porous body 87a does not exist (a part in which only the first space portion 85a exists) in a part of the front-rear direction.

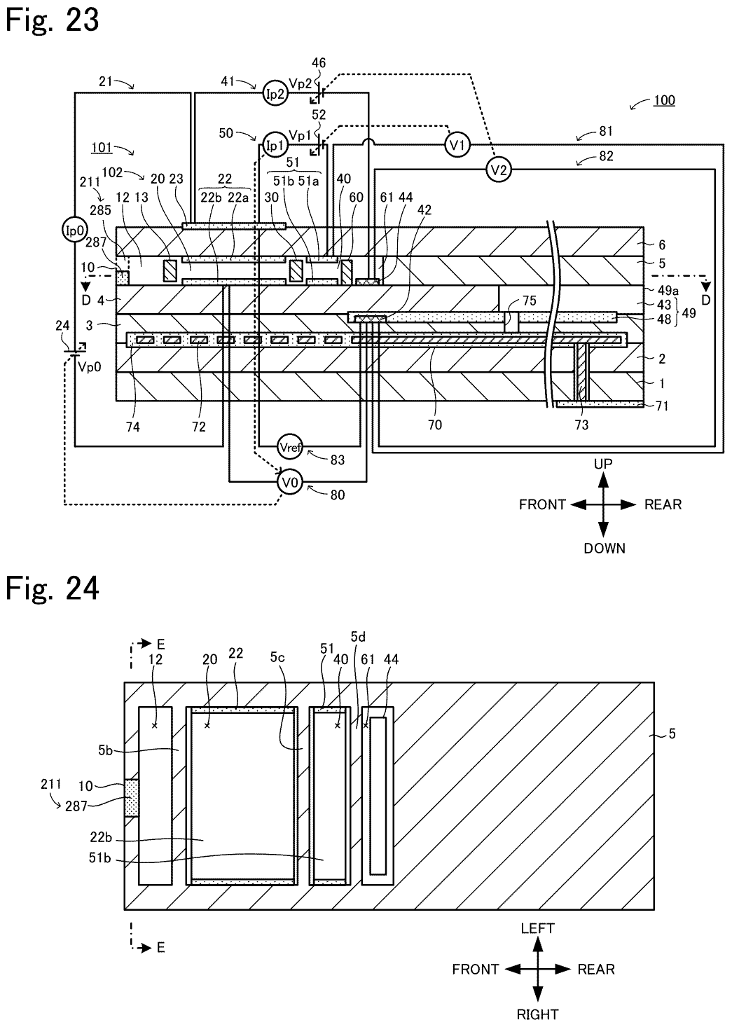

In the embodiment described above, the first diffusion control section 11 has the diffusion control section 11a provided between the spacer layer 5 and the second solid electrolyte layer 6 and the diffusion control section 11b provided between the spacer layer 5 and the second solid electrolyte layer 6, but the first diffusion control section 11 is not limited to this embodiment, and the first diffusion control section 11 may have only one of the diffusion control sections 11a and 11b. The diffusion control section 11a is provided between the spacer layer 5 and the second solid electrolyte layer 6 that are formed adjacent to each other, but the diffusion control section 11a is not limited to this embodiment. For example, the diffusion control section 11a may be a space formed by punching out any of the layers constituting the element body 102. For example, the sensor element 101 may include a first diffusion control section 211 illustrated in FIGS. 23 to 25 instead of the first diffusion control section 11. As illustrated in FIGS. 23 to 25, the first diffusion control section 211 is formed by punching out the spacer layer 5 in the up-down direction. Since the first diffusion control section 211 has a longer length in the up-down direction than the diffusion control section 11a of the first diffusion control section 11, the length in the left-right direction is made shorter than that of the diffusion control section 11a so that an area of a cross-section of the first diffusion control section 211 perpendicular to the front-rear direction approximately becomes the same as that of the diffusion control section 11. In addition, the left-right length of the first diffusion control section 211 is shorter than the left-right length of the buffer space 12 (see FIG. 24).

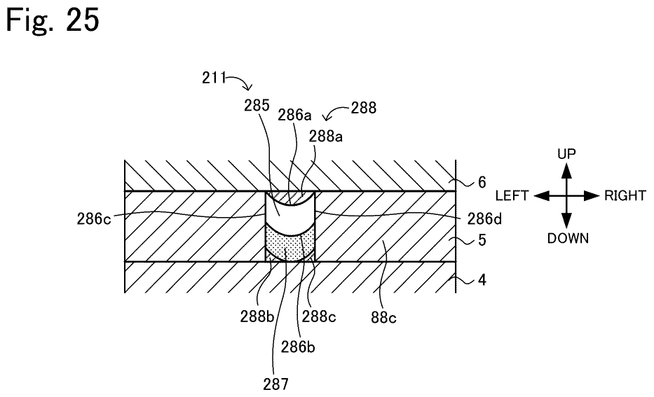

The first diffusion control section 211 has a space portion 285 and a porous body 287. As illustrated in FIG. 25, the space portion 285 and the porous body 287 both each have a cross-section perpendicular to the front-rear direction that has a shape of a rectangle with curved upper side and curved lower side. Specifically, in the cross-section perpendicular to the front-rear direction, the space portion 285 has first to fourth portions 286a to 286d as an outer circumference of the cross-section. The first portion 286a, the second portion 286b, the third portion 286c, and the fourth portion 286d correspond to the upper, lower, left, and right sides of the outer circumference of the cross-section of the space portion 285. The first portion 286a and the second portion 286b face each other in the up-down direction (the layered direction) and extend along the left-right direction (the second direction), respectively. The third portion 286c and the fourth portion 286d face each other in the left-right direction and extend along the up-down direction, respectively. The first part 286a and the second part 286b are shaped similarly to the first part 86a and the second part 86b in FIG. 8. Specifically, the first part 286a and the second part 286b each have a curved shape and are each curved to form a convex shape on the same side in the up-down direction (in this case, lower side). The third portion 286c and the fourth portion 286d each have a straight line shape along the up-down direction, similarly to the shapes of the third portion 86c and the fourth portion 86d in FIG. 8. The porous body 287 also has the same shape as the space portion 285. In addition, the entire of the first diffusion control section 211 also has a cross-section perpendicular to the front-rear direction that has a shape of a rectangle with curved upper side and curved lower side, similar to the space portion 285 and the porous body 287. The shape of the upper and lower sides of the first diffusion control section 211, that is, the shape of the first portion 286a of the space portion 285 and the lower side of the porous body 287, can be adjusted by the shapes of a first to third structural portions 288a to 288c that a structural portion 288 includes, similarly to FIG. 8. Specifically, the first structural portion 288a is provided on the lower surface of the second solid electrolyte layer 6. The first structural portion 288a is thin at both left and right ends in the cross-section perpendicular to the front-rear direction and becomes thicker toward a center in the left-right direction. With this configuration, a lower surface of the first structural portion 288a thus curves so that the lower surface has a convex shape to the down direction, thereby constituting the first portion 286a of the space portion 285. The second structural portion 288b and the third structural portion 288c are provided on the upper surface of the first solid electrolyte layer 4. The second structural portion 288b is thick at left end in the cross-section perpendicular to the front-rear direction and becomes thinner toward a center of the space portion 285 in the left-right direction. The third structural portion 88c is thick at right end in the cross-section perpendicular to the front-rear direction and becomes thinner toward a center of the space portion 285 in the left-right direction. With these configurations, an upper surface of the second structural portion 288b and an upper surface of the third structural portion 288c as a whole are curved so that the upper surfaces have a convex shape to the down direction, thereby forming the shape of lower side of the porous body 287. In FIG. 23, the structural portion 288 is omitted from the illustration. Left and right sides of the first diffusion control section 211 (including the third portion 286c and the fourth portion 286d of the space portion 285) that each have the straight line shape are constituted by not structural portion 288, but are formed as inner peripheral faces (side surfaces) of punch holes provided in the spacer layer 5 (holes penetrating the spacer layer 5 in the up-down direction). This first diffusion control section 211 has the space portion 285 and the porous body 287, similar to the diffusion control section 11a in the embodiment described above, so that it is possible to make a flow velocity of the measurement-object gas passing through the porous body 287 different from a flow velocity of the measurement-object gas passing through the space portion 285, thereby it is possible to cause turbulence in the measurement-object gas after passing through first diffusion control section 211 and to increase a pressure loss of the measurement-object gas by the turbulence of the measurement-object gas. As in the first diffusion control section 11a in FIG. 8 described above, in the space portion 285 of this first diffusion control section 211, the first portion 286a and the second portion 286b of the cross-section perpendicular to the front-rear direction each have the curved shape. With this configuration, the shape of this cross-section is the predetermined shape described above (the shape with a longer outer circumference than at least one of the reference rectangles S1 to S3). As a result, a hydraulic diameter of the space portion 285 becomes smaller than a hydraulic diameter of at least one of the reference rectangles S1 to S3, and thus the pressure loss of the measurement-object gas passing through the space portion 285 becomes higher. Therefore, the same effect as that of the first space portion 85a in FIG. 8 described above can be obtained. The shapes of the space portion 285 and the porous body 287 may adopt any of the various embodiments of the first space portion 85a and the porous body 87a described above.

In the embodiment described above, the first diffusion control section 11 has the first and second porous bodies 87a and 87b and the first and second space portions 85a and 85b, but the present invention is not limited to this, and one or more of the first diffusion control section 11, the second diffusion control section 13, the third diffusion control section 30, and the fourth diffusion control section 60 may have a porous body and a space portion.

In the embodiment described above, each of the front ends of the first diffusion control section 11 is a gas inlet 10, but the present invention is not limited to this. For example, the first bridging portion 5a and the first diffusion control section 11 may be disposed behind the gas inlet 10 in the measurement-object gas flow section.

In the embodiment described above and FIG. 23, the first diffusion control sections 11 and 211 allow the measurement-object gas to flow in the front-rear direction, but the present invention is not limited to this. For example, the gas inlet 10 may be opened on the left or right surface of the element body 102, and the first diffusion control section 11 may allow the measurement-object gas to flow in the left-right direction. In this case, the left-right direction corresponds to the first direction, and the front-rear direction corresponds to the second direction.

In the embodiment described above, the outer pump electrode 23 plays a role as the electrode (also referred to as an outer main pump electrode) to be paired with the inner pump electrode 22 in the main pump cell 21, plays a role as the electrode (also referred to as an outer auxiliary pump electrode) to be paired with the auxiliary pump electrode 51 in the auxiliary pump cell 50, and plays a role as the electrode (also referred to as an outer measurement electrode) to be paired with the measurement electrode 44 in the measurement pump cell 41; however, the present invention is not limited thereto. One or more of the outer main pump electrodes, the outer auxiliary pump electrode, and the outer measurement electrode may be provided separately from the outer pump electrode 23 on outer surface of the element body 102 so as to be in contact with the measurement-object gas.

In the embodiment described above, the outer pump electrode 23 is exposed outside the sensor element 101, but the present invention is not limited to this, it is sufficient if the outer pump electrode 23 is provided on the outer surface of the element body 102 so as to be in contact with the measurement-object gas. For example, the sensor element 101 may include a porous protective layer that covers the element body 102 and allows the measurement-object gas to pass through, and the outer pump electrode 23 may also be covered by the porous protective layer.

While the sensor element 101 according to the above embodiment is configured to detect the concentration of NOx in the measurement-object gas, such an embodiment is not limited as long as the sensor element is configured to detect the concentration of any specific gas in the measurement-object gas. For example, the concentration of any oxide other than NOx may be defined as the specific gas concentration. If the specific gas is an oxide, oxygen is produced when the specific gas itself is reduced in the third internal cavity 61, as with the case of the above embodiment. Therefore, the measurement pump cell 41 can detect the specific gas concentration by acquiring a value (for example, the pump current Ip2) detected in correspondence with the oxygen. Alternatively, the specific gas may be a non-oxide, such as ammonia. If the specific gas is a non-oxide, the specific gas is converted into an oxide (e.g., into NO in the case of ammonia), so that oxygen is produced when the converted gas is reduced in the third internal cavity 61. Thus, the measurement pump cell 41 can acquire a value (e.g., the pump current Ip2) detected in correspondence with this oxygen and thus detect the specific gas concentration. For example, since the inner pump electrode 22 in the first internal cavity 20 functions as a catalyst, the ammonia is converted into NO in the first internal cavity 20.

In the above-described embodiment, the element body 102 of the sensor element 101 is a layered body having a plurality of solid electrolyte layers (layers 1 to 6), but is not limited thereto. The element body 102 of the sensor element 101 may include at least one oxygen-ion-conductive solid electrolyte layer. For example, in FIG. 1, the layers 1 to 5 other than the second solid electrolyte layer 6 may be layers (e.g., layers composed of alumina) composed of a material other than solid electrolyte. In this case, the electrodes of the sensor element 101 may be disposed in the second solid electrolyte layer 6. For example, the measurement electrode 44 in FIG. 1 may be disposed on the lower surface of the second solid electrolyte layer 6. Also, the reference gas inlet space 43 may be provided in the spacer layer 5 instead of the first solid electrolyte layer 4, the reference gas inlet layer 48 may be provided between the second solid electrolyte layer 6 and the spacer layer 5 instead of between the first solid electrolyte layer 4 and the third substrate layer 3, and the reference electrode 42 may be provided rearward of the third internal cavity 61 and on the lower surface of the second solid electrolyte layer 6.

The present application claims priority from Japanese Patent Application No. 2024-095787 filed Jun. 13, 2024, the entire contents of which are incorporated herein by reference.

What is claimed is:

1. A sensor element for detecting a concentration of a specific gas in a measurement-object gas, the sensor element comprising:
an element body having an oxygen-ion-conductive solid electrolyte layer, and having a measurement-object gas flow section inside that introduces the measurement-object gas from a gas inlet and causes the measurement-object gas to flow in a first direction;
a pump cell including an inner electrode provided in an internal cavity of the measurement-object gas flow section and an outer electrode provided on an outer surface of the element body, and
a diffusion control section that is located upstream of the internal cavity in the measurement-object gas flow section and that provides the measurement-object gas introduced from the gas inlet with diffusion resistance,
wherein the diffusion control section includes a porous body and a space portion that are provided in at least a part of the diffusion control section in the first direction, the porous body is provided so as to occupy a part of a cross-section of the diffusion control section perpendicular to the first direction and allows the measurement-object gas to pass therethrough, and the space portion includes a part of the cross-section not occupied by the porous body and allows the measurement-object gas to pass therethrough.

2. The sensor element according to claim 1,
wherein the porous body is provided in the diffusion control section so as to occupy 30% to 70% of the cross-section.

3. The sensor element according to claim 1,
wherein a porosity of the porous body is 15% or less.

4. The sensor element according to claim 1,
wherein the porous body is provided in a plurality so as to form parallel flow paths in the cross-section.

5. The sensor element according to claim 1,
wherein the element body has a longitudinal direction, and is a layered body that is composed of multiple layers stacked in a layered direction perpendicular to the longitudinal direction,
a shape of the cross-section of the space portion is a predetermined shape with a longer outer circumference than a reference rectangle, and
the reference rectangle is a rectangle that has the same area as the cross-section of the space portion, and the reference rectangle is a rectangle that is formed by deforming the circumscribed rectangle of the cross-section, which is composed of two sides parallel to the layered direction and two sides parallel to a second direction that is perpendicular to the layered direction, so that short sides become shorter while maintaining the length of long sides, or a rectangle that is formed by deforming the circumscribed rectangle while maintaining a ratio of each side, or a rectangle that is formed by changing the length of each side of the circumscribed rectangle so as to maximize the overlapping area with the cross-section.

6. The sensor element according to claim 5,
wherein the outer circumference of the predetermined shape includes a first portion and a second portion that face each other in the layered direction and extend along the second direction, respectively, and
at least a part of the first portion and/or at least a part of the second portion each have a curved shape.

7. The sensor element according to claim 5,
wherein the outer circumference of the predetermined shape includes a first portion and a second portion that face each other in the layered direction and extend along the second direction, respectively, and
at least a part of the first portion and/or at least a part of the second portion each have a polygonal line shape.

8. The sensor element according to claim 6,
wherein the first portion and the second portion each have a convex shape on the same side in the layered direction.

9. The sensor element according to claim 6,
wherein the first portion and the second portion each have a convex shape toward a side where they approach each other or toward a side where they are separated from each other in the layered direction.

10. The sensor element according to claim 5,
wherein the diffusion control section has a plurality of the space portions, and
each of the plurality of the space sections has a shape of the cross-section that is the predetermined shape.

11. The sensor element according to claim 1,
wherein the diffusion control section is formed such that a cross-sectional area of a cross-section perpendicular to the first direction is smaller in at least a part of a downstream side of an upstream end than in the upstream end.

12. The sensor element according to claim 11,
wherein the diffusion control section has an outline of cross-section parallel to the first direction, and the outline extends so as to curve with respect to the first direction.

13. A gas sensor comprising the sensor element according to claim 1.
